(19) United States
(12) Patent Application Publication
(10) Pub. No.: US 2007/0197938 A1
(43) Pub. Date: Aug. 23, 2007

(54) PERFORMANCE TRACKING SYSTEMS AND METHODS

(76) Inventors: WILLIAM RANDALL TYSON, Jamestown, RI (US); JOHN HEANEY, Warwick, RI (US); DANIEL M. LABY, Sharon, MA (US); DAVID DURFEE, North Scituate, RI (US); DAVID S. BANKS, Cranston, RI (US); DANIEL BENJAMIN, Providence, RI (US)

Correspondence Address: LOWRIE, LANDO & ANASTASI, RIVERFRONT OFFICE, ONE MAIN STREET, ELEVENTH FLOOR, CAMBRIDGE, MA, 02142, US

(21) Appl. No.: 11/610,695

(22) Filed: Dec. 14, 2006

Related U.S. Application Data
(60) Provisional application No. 60/750,134, filed on Dec. 14, 2005.

(51) Int. Cl. A61B 5/103; A61B 5/117

(52) U.S. Cl. 600/587; 600/595

(57) ABSTRACT

In one aspect, the invention provides a system for determining lower body strength of a subject. In one embodiment, the system includes an electronic device having a plurality of sensors distributed in a predetermined array and a frame coupled to the electronic device and configured to support the device to allow the subject to activate one sensor of the plurality of sensors.

### CROSS-REFERENCE TO RELATED APPLICATIONS

This application claims priority under 35 U.S.C. §119(e), to U.S. Provisional Patent Application No. 60/750,134, filed on Dec. 14, 2005, the contents of which is hereby incorporated herein by reference in its entirety.

### BACKGROUND OF INVENTION

1. Field of Invention

The present invention is generally related to physical skills assessment and, more particularly, is related to physical performance test systems and methods.

2. Discussion of Related Art

Physical skill tests may be used to evaluate athletic skills, occupational skills, among others. Most physical skill tests rely primarily on manual or semi-automated test procedures that use equipment and protocols that are subjective and fraught with deviation or systemic errors. Using such equipment and/or procedures may result in inconsistent evaluations, resulting in a lack of standardized, repeatable and reproducible data, especially when comparing such evaluations over multiple locations. The human component of many existing physical testing processes is also susceptible to overt or inadvertent assistance by the test evaluator.

### SUMMARY OF INVENTION

Preferred embodiments of a system and method are disclosed. One embodiment of a method, among other embodiments, includes receiving standardized physical performance test data over a network from a test site, the standardized physical performance test data corresponding to physical performance for a plurality of individuals, and processing the standardized physical performance test data to provide standardized data of physical performance among the plurality of individuals.

In one aspect, the invention provides a system for determining lower body strength of a subject. In one embodiment, the system includes an electronic device having a plurality of sensors distributed in a predetermined array and a frame coupled to the electronic device and configured to support the device to allow the subject to activate one sensor of the plurality of sensors.

In another aspect, the invention provides a method of determining lower body strength of a subject where the method includes acts of distributing a plurality of sensors included in an electronic device in a predetermined array, detecting activation of one of the plurality of sensors by the subject and providing a lower body test result for the subject.

In a further aspect, the invention provides a system for determining upper body strength of a subject. In one embodiment, the system includes an object upon which a force is exerted by the subject during a strength test of the subject, a frame, a force detector positionable on the frame to receive the object during the test and a controller coupled to the force detector and configured to determine a value related to kinetic energy imparted on the force detector by the object during the test.

In yet another aspect, the invention provides a method of determining an upper body strength of a subject where the method includes acts of adjusting to a testing position a force detector configured to receive an object upon which a force is exerted by the subject during a strength test of the subject, wherein the testing position is established, at least in part, based on a size of the subject. According to one embodiment, the method also includes acts of detecting a force exerted by the subject and providing an upper body strength test result for the subject. In a further embodiment, the detected force is determined from data corresponding to an impact force of the object on the force detector.

In a still further aspect, the invention provides a system for evaluating at least one of a participant's reaction time or a participant's eye-hand coordination. In one embodiment, the system includes a workstation having a processor, a display and a user input device, wherein the processor is programmed to present one or more objects on the screen, measure a participant's response to presentation of the objects and determine a score for the participant. In a further embodiment, the system includes a communication device that communicates the score to a central device in a test facility.

In still another aspect, the invention provides a method for evaluating a participant's reaction time where the method includes acts of: (a) displaying an object; (b) recording an input by the participant following act (a); (c) determining an elapsed time between a time of occurrence of act (a) and a time of occurrence of the input; (d) repeating acts (a)-(c) for a plurality of objects; and (e) determining a score based on the elapsed time determined at act (d) for each of the plurality of objects.

In another aspect, the invention provides a method for evaluating a participant's eye-hand coordination where the method includes acts of: (a) displaying, in a display, a first object and a second object; (b) allowing a location of the second object in the display to be controlled by the participant; (c) randomly moving a location of the first object in the display; (d) collecting data, for a plurality of points in time, representative of a distance between the first object and the second object as the participant moves the location of the second object in an attempt to maintain a spatial relationship between the first object and the second object; (e) performing regression analysis on the data; (f) performing an analysis of a variability of the data; (g) comparing the results of act (f) with benchmark data; and (h) determining a score based on the results of act (e) and act (g).

Other systems, methods, features, and advantages will be or become apparent to one with skill in the art upon examination of the following drawings and detailed description. It is intended that all such additional systems, methods, features, and advantages be included within this description and be within the scope of the disclosure.

### BRIEF DESCRIPTION OF DRAWINGS

Many aspects of embodiments of a system and method can be better understood with reference to the following drawings. The components in the drawings are not necessarily to scale, emphasis instead being placed upon clearly illustrating the principles of systems and methods. Moreover, in the drawings, like reference numerals designate corresponding parts throughout the several views.

FIG. 1 is a block diagram of a performance tracking system.

FIG. 2A is a schematic diagram depicting an embodiment of the performance tracking system shown in FIG. 1.

FIG. 2B is a block diagram of an embodiment of a central server that can implement performance tracking software of the performance tracking system shown in FIG. 1.

FIGS. 2C-2I are flow diagrams that depict method embodiments of the performance tracking software shown in FIG. 2B.

FIGS. 3A-3C are block diagrams that illustrate functionality provided by web-interface screens provided by the performance tracking software shown in FIG. 2B.

FIG. 4A is a schematic diagram that depicts an embodiment of a test station in front elevation view as shown in FIG. 2A.

FIG. 4B is a schematic diagram that depicts a side view of the test station shown in FIG. 4A.

FIG. 4C is a schematic diagram that depicts an embodiment of a controller in front elevation view used in the test station shown in FIGS. 4A-4B.

FIG. 4D is a schematic diagram that depicts a side view of the controller shown in FIG. 4C.

FIG. 4E is a flow diagram that depicts a method embodiment 400a of the test station module 400 shown in FIGS. 4A-4B.

FIG. 5 is a block diagram that illustrates functionality for a web-interface embodiment provided by the performance testing software for a server located at a performance lab as shown in FIG. 1.

FIG. 6 is a schematic diagram that depicts one embodiment of a performance lab as shown in FIG. 1.

FIGS. 7A-7H are schematic diagrams that illustrate exemplary test stations illustrated in FIG. 6.

FIG. 8 is a schematic diagram of a lower body strength test station in accordance with one embodiment.

FIG. 9 is a perspective view of a measurement device used in the test station of FIG. 8.

FIG. 10 is a side view of a keyboard device used in the measurement device of FIG. 9.

FIG. 11 is a side view of the keyboard device of FIG. 10 with a side cover removed.

FIGS. 12A-12B are cross-sectional end views of the keyboard device of FIG. 10 in accordance with a first and second embodiment, respectively.

FIG. 13 is a functional block diagram of the test station of FIG. 8.

FIG. 14 is a schematic diagram of an upper body strength test station in accordance with one embodiment.

FIG. 15 is a functional block diagram of the upper body strength station of FIG. 14.

FIG. 16 is a graph showing a voltage waveform representative of a measured force in the test station of FIG. 14.

FIG. 17 is a schematic diagram of an additional test station used in embodiments of the invention.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

Preferred embodiments of a performance tracking system and method (herein referred to simply as a performance tracking system) are disclosed. A performance tracking system include mechanisms for quantifying assessment of individuals' physical skills using networked automatic measuring devices, software, and/or hardware. In a performance tracking system, physical skills evaluations are assessed and recorded using secure proprietary networked testing and methodology, software and equipment in one or more authorized physical performance laboratories. The skills data is thereafter transmitted from a performance laboratory (herein, performance lab) over a network, and processed with performance tracking software to create a relational database that can be sorted by numerous criteria. The data can be automatically processed and compiled into a statistical numerical comparative score, or a distinct set of numerical values, in a secure computer database. For example, data may be inputted into a membership computer database which is configured with performance tracking software to compute and create a score or a set of numerical values that can be used for comparison purposes within defined parameters or standards.

A performance tracking system includes, but is not limited to, performance tracking software accessible on the World Wide Web (Internet) that allows for interaction of member participants and certified testing organizations. Performance tracking software and/or hardware is packaged into a protocol that is repeatable and reproducible in multiple authorized locations, which enables a member participant to be evaluated and compared on a quantitatively and statistically valid method.

A performance tracking system provides for the exchange of information between students or other individuals and academic and/or occupational institutions, providing methodologies and resources to quantify the assessment of physical prowess for comparison and improvement. A performance tracking system provides a method for a testing organization to generate useful comparative data and can provide an additional source of funding for athletic or occupational programs. A performance tracking system can be used to assess large and/or fine motor skills. A performance tracking system provides the ability to quantify the physical attributes of individuals, which is useful for personal development, admissions evaluations for college and high school athletic programs, as well as evaluations for occupational programs. A performance tracking system can be a resource to the institutions and the member participants (e.g., individual student athletes, candidates or potential candidates for various occupational positions, etc.) as the database can serve as a communications center for the exchange of data for both member participants and member institutions. These assessments can be used for indicators of potential success in occupations that demand physical skill (such as firemen, police etc.) and/or specific eye-hand coordination (such as dentist, pilot, laser surgeon etc.).

A performance tracking system can enable the development of programs to assist in the evaluation of physically challenged individuals. This program may incorporate performance tracking methodology as an outreach to provide opportunities for career placement for the physically challenged.

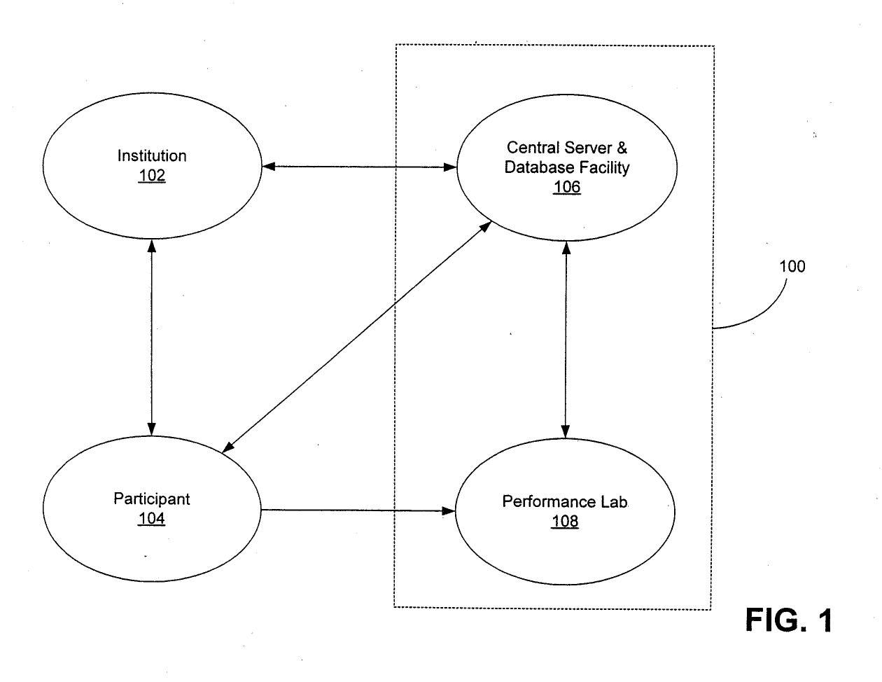

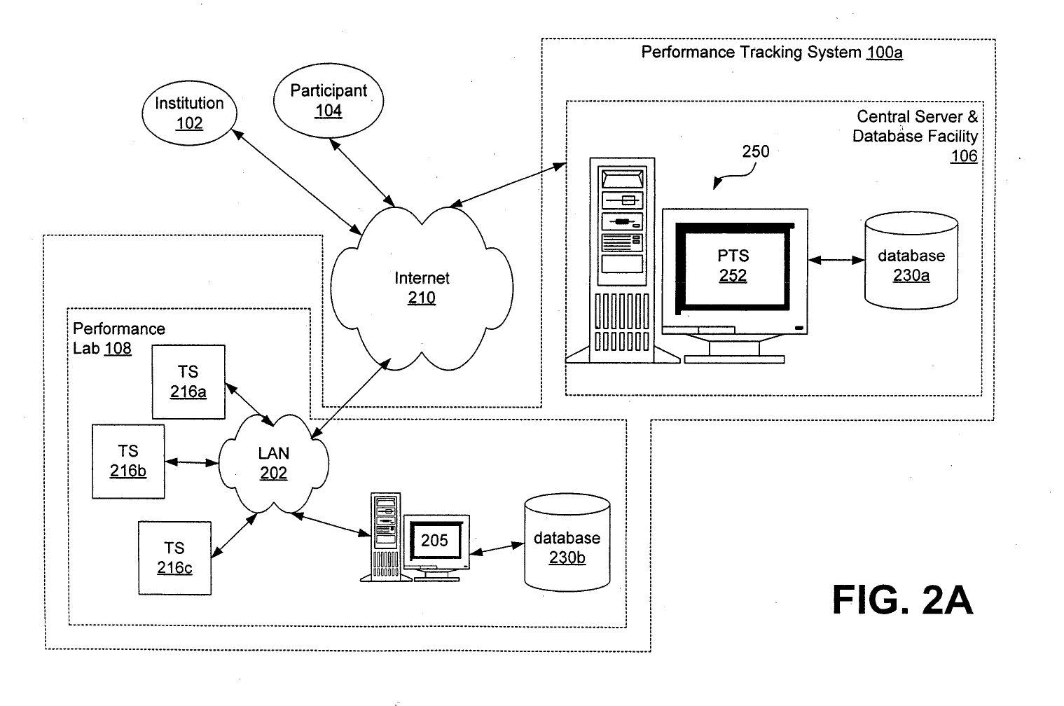

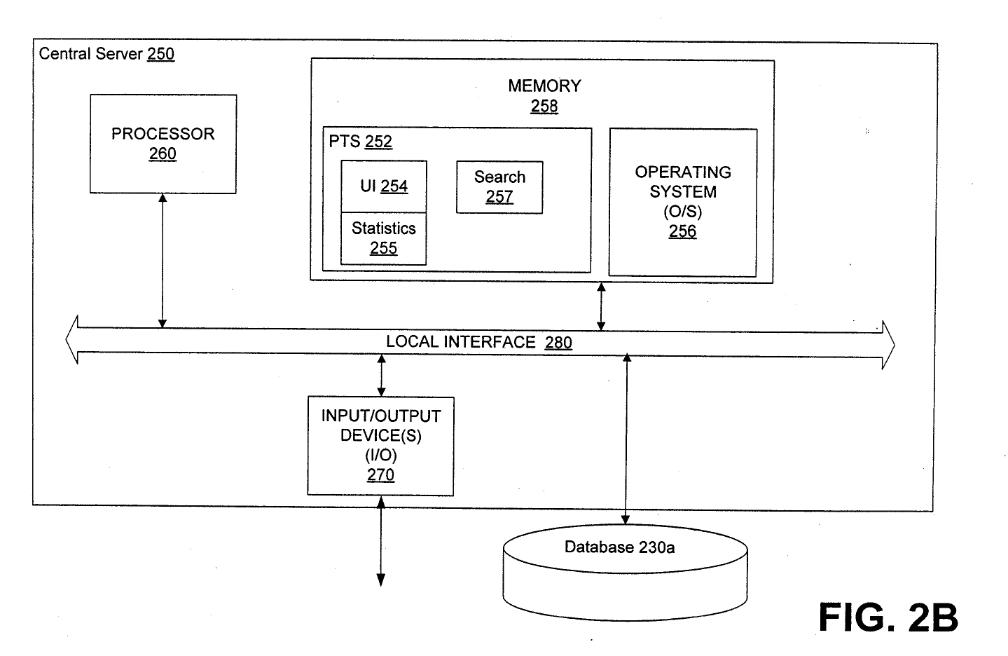

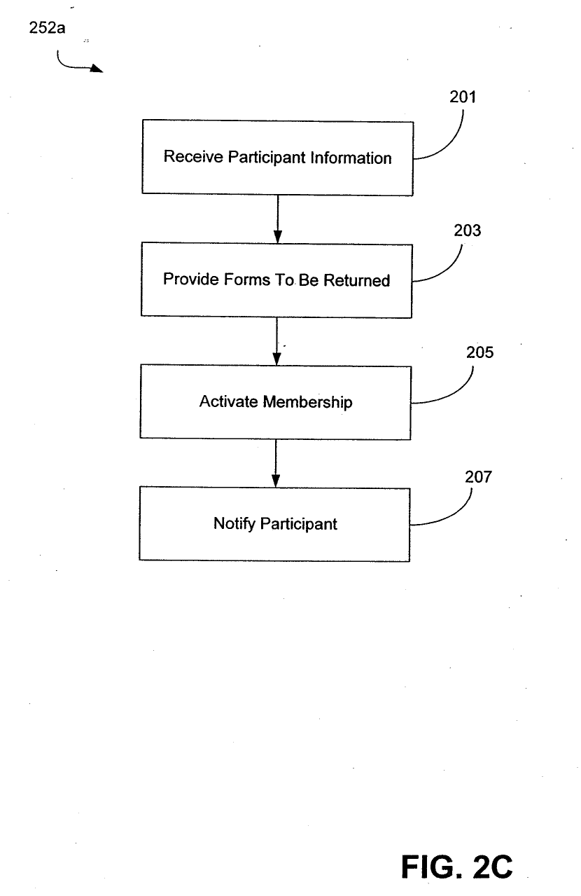

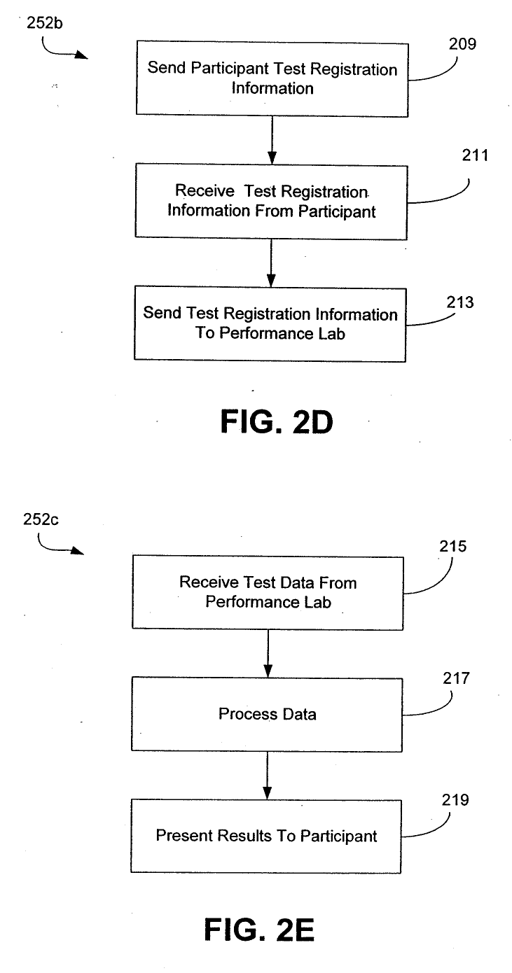

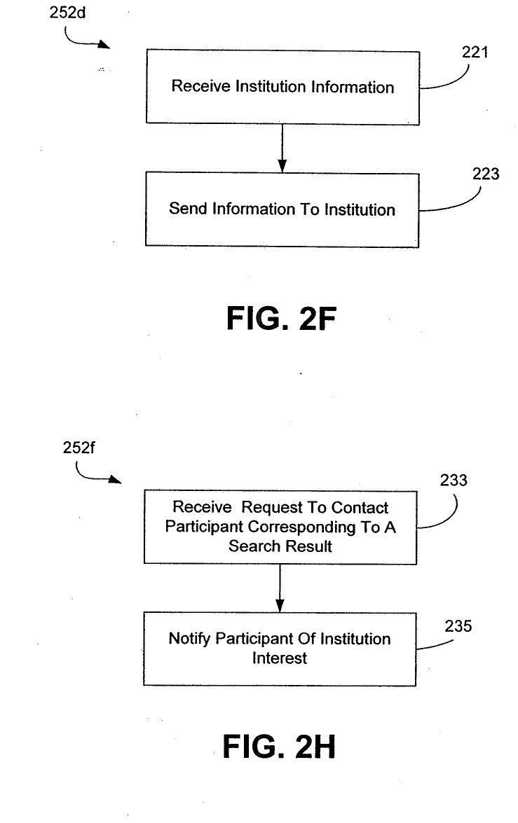

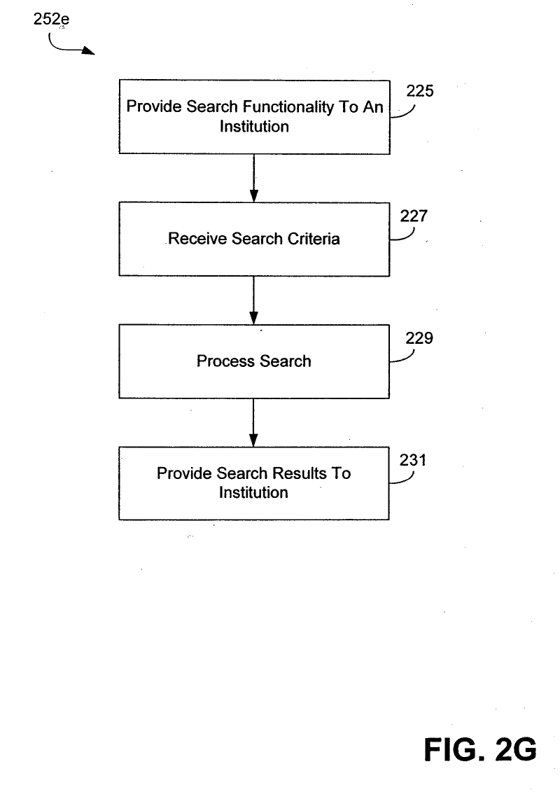

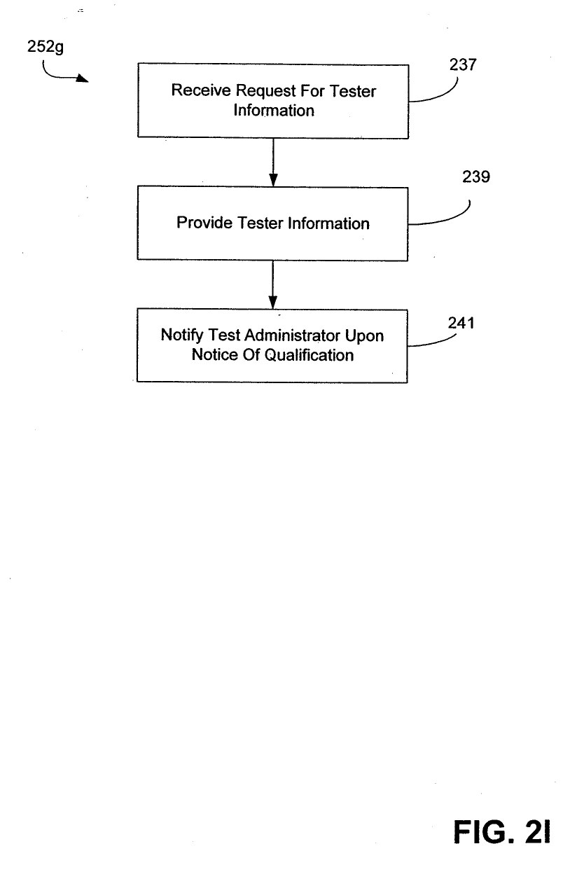

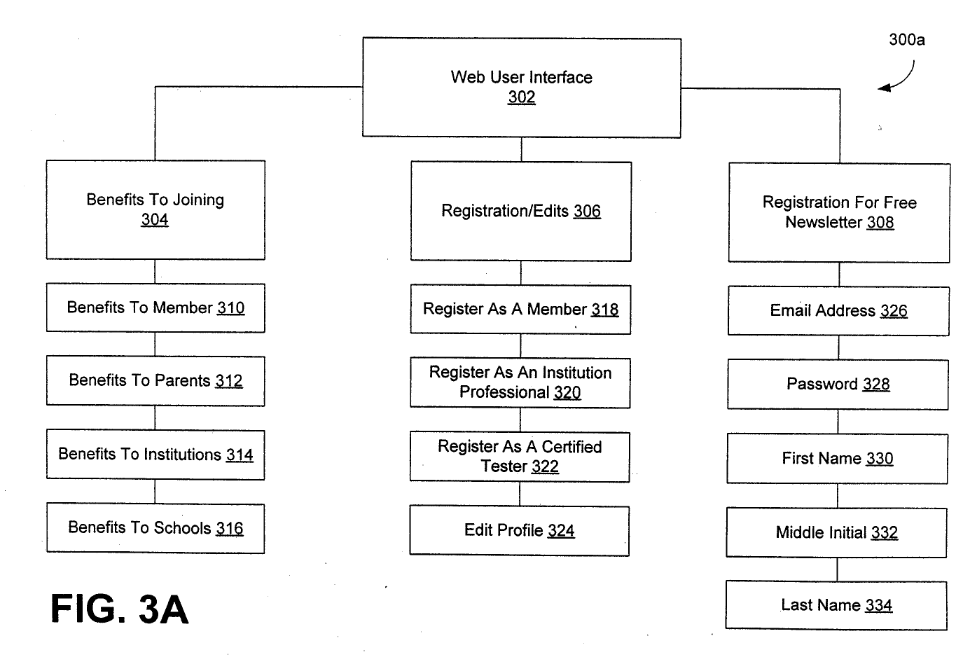

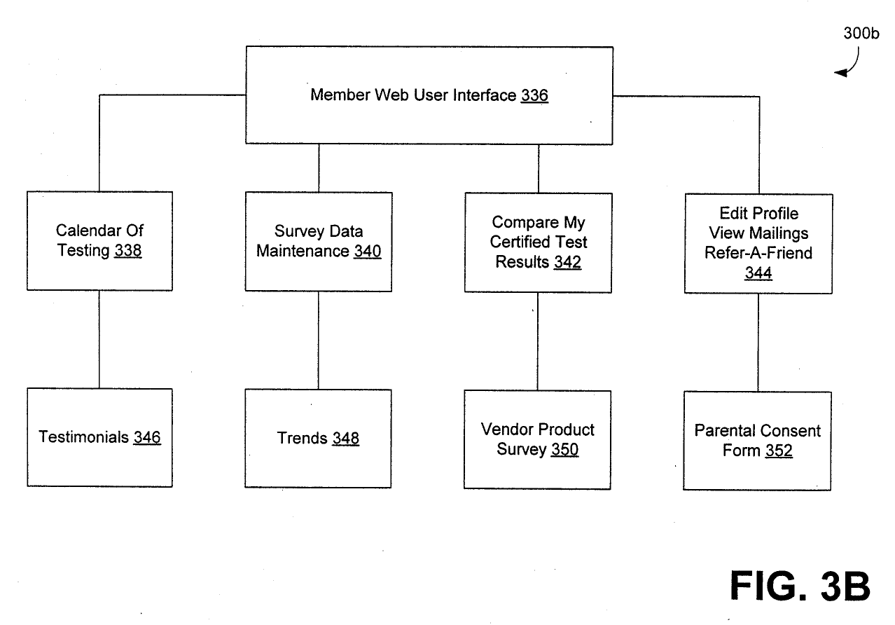

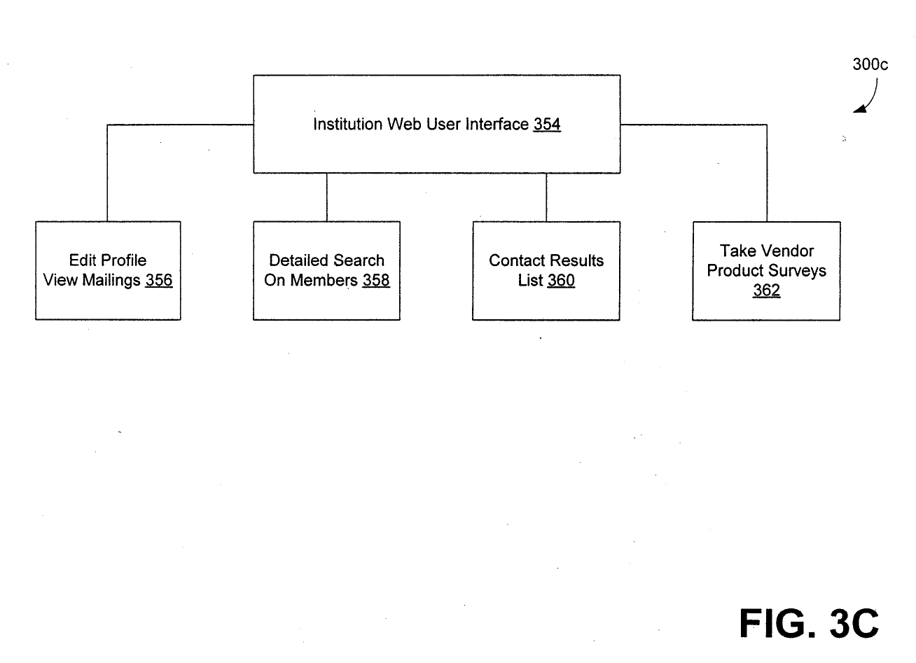

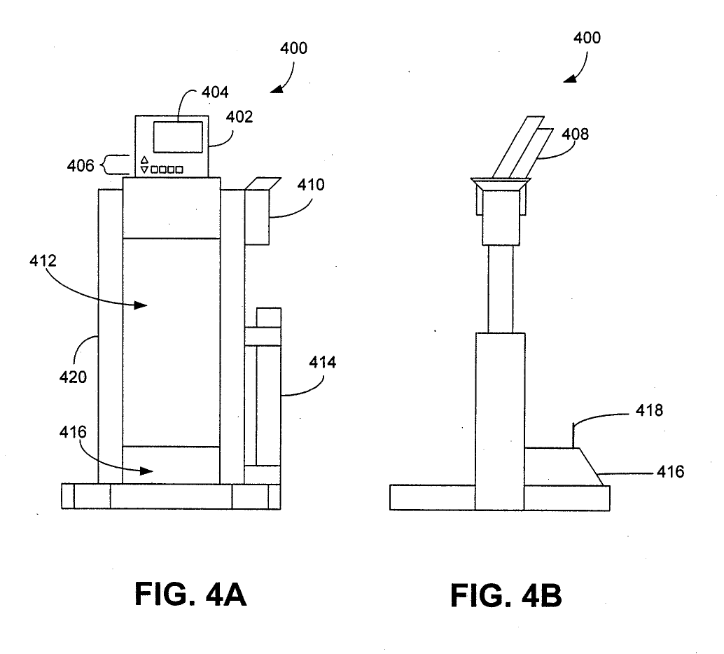

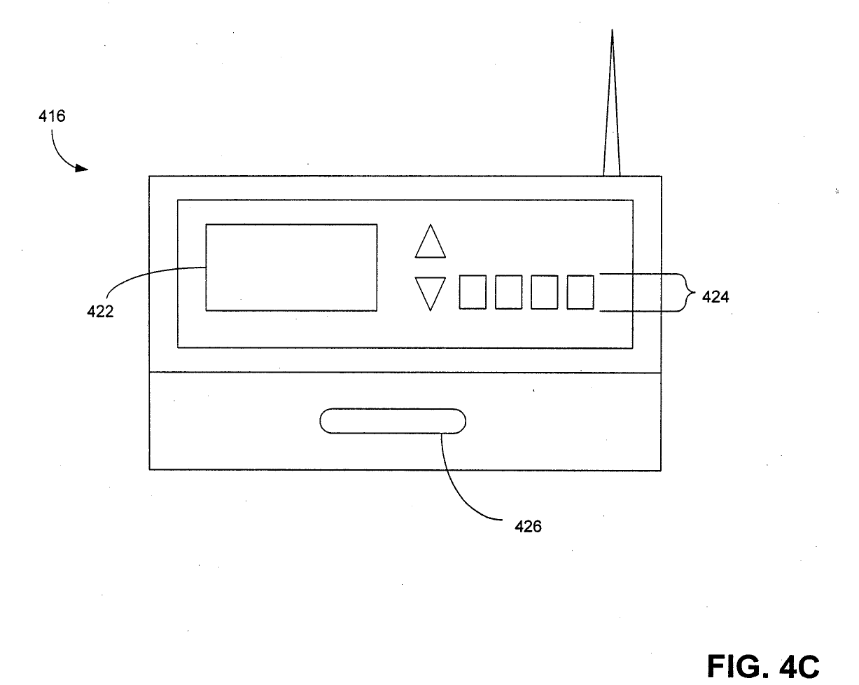

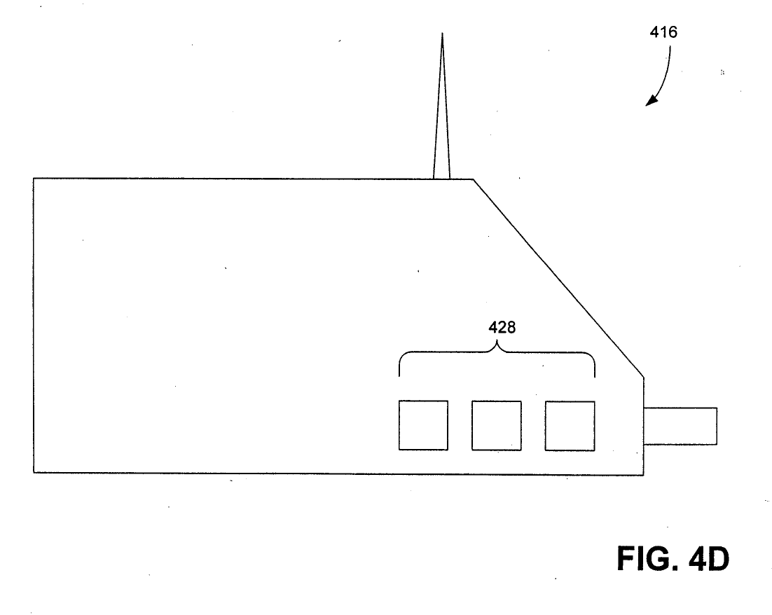

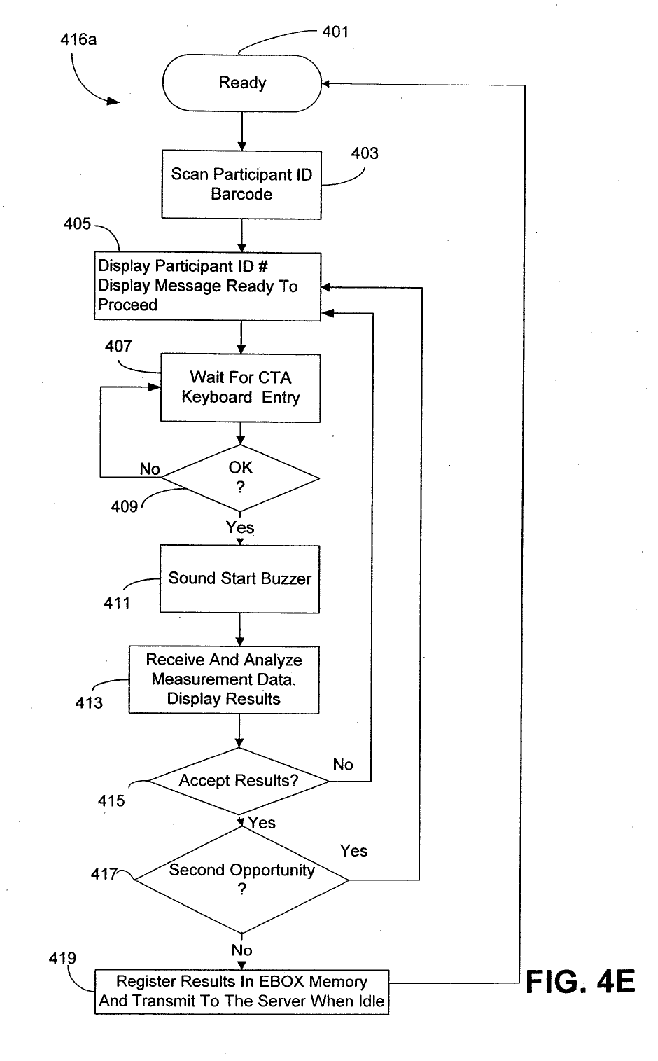

Preferred embodiments of a performance tracking system is described in association with FIG. 1, along with components and software methods as illustrated in FIGS. 2A-3C as implemented for athletic physical skills assessments. An example performance lab and components and associated methods are illustrated in FIGS. 4A-7H. It will be understood that the disclosed systems and methods also encompass in scope other physical skill assessment implementations, such as occupational skills testing, among others.

FIG. 1 illustrates an embodiment of a performance tracking system 100. The performance tracking system 100 includes a central server and database facility 106 and one or more performance labs 108. The performance tracking system 100 interacts with one or more member institutions 102 and one or more member participants 104. The use of the term “member” indicates subscription to the performance tracking system 100 by an individual, institution, or other entity, although non-subscribers may also interact with limited access to the performance tracking system 100. Although shown as single entities, it will be understood that a plurality of member participants 104 and/or member institutions 102 can exist in and operate in cooperation with the performance tracking system 100. A participant (also known as an applicant) that is interested in becoming a member of the performance tracking system 100 can begin by opening a World Wide Web site (herein, simply web-site) provided for and operated by the central server and database facility 106. Membership in the performance tracking system 100 includes access to a web-based program to register into the performance tracking system 100. The performance tracking system 100 includes a practice regiment and other information to prepare for one or more certified tests that evaluate various physical performance criteria, whether athletic, vocational, etc.

The central server and database facility 106 includes functionality to compare peer group certified test results and serve as a communication center for the transfer of member participants' certified test results, demographic information, and academic preferences to selected institutions of the member participants' choice. A web-site provided by the central server and database facility 106 can explain the program and the processes needed to participate. If a participant decides to join as a member (and thus becomes a member participant 104) of the performance tracking system 100, payment of a membership fee(s), such as via secure transactions, is required. After payment of the membership fee, a member participant 104 is issued an individualized membership number, which is the identifier of the participant for all further interactions. The member participant 104 can receive electronic transmissions via a web-site (or other mechanisms of information transfer) of information and opportunities that are included in the membership program.

If the member participant 104 decides to attend a testing session at an authorized performance lab 108, the locations and the dates of the test sites may be found on a web-site provided by the central server and database facility 106. Registration and confirmation for a test can be conducted via a web-site.

A web-site provided by the central server and database facility 106 may serve as a coordination center of the performance tracking system 100. One or more databases of the central server and database facility 106 can be based on software that functions to collect, receive, manipulate, analyze, process, compare, and/or communicate data that is inputted through the web-site and other secure resources.

A member institution 102 may include an organization that has an interest in receiving data that has been released by the member participant 104. The member institution 102 may include an academic institution, occupational organization, and/or a government agency. Thus, an institution can pay for a subscription (to become a member institution 102) to participate in the performance tracking system 100 and be allowed to query one or more databases (provided by the central server and database facility 106) in search of candidates or applicants that fit specialized criteria that has been submitted in a proscribed format. The criteria can be analyzed by software of the central server and database facility 106 using data in the aforementioned database(s). One or more member participants 104 can automatically receive a communication from the central server and database facility 106 that a specific member institution 102 has requested information about a member participant 104 that possess some or all of the characteristics that have been recorded in a collection of the data obtained from testing at a performance lab 108. The member institution 102 preferably does not receive any identifying reports on the member participant 104 that meets the criteria that the institution 102 has selected. Instead, the member participant 104 is given contact information for the member institution 102 that has expressed an interest, leaving it to the member participant 104 to contact the member institution 102.

There are preferably many performance labs 108 that are located, for example, on a geographical basis. The performance lab 108 is preferably authorized and licensed by administrators or authorized representatives of the performance tracking system 100. For an athletic performance-based performance tracking system 100, the performance lab 108 preferably conducts testing protocols that include but are not limited to those that measure body composition, endurance, speed/acceleration, muscle explosion/power, and agility/flexibility, although not limited as such. The performance lab 108 may be equipped with proprietary equipment and procedures used to conduct a testing program in a standardized manner with authorized, certified and trained evaluators. The performance lab 108 includes equipment that enables transmission of data to and from one or more databases of the central server and database facility 106.

FIG. 2A is a schematic diagram depicting one embodiment of a performance tracking system 100a. The performance tracking system 100a includes one or more of the following: a central server and database facility 106 and one or more performance labs 108. The performance tracking system 100a interacts with one or more member participants 104 and member institutions 102 over a medium, such as the Internet 210. The performance lab 108 includes one or more local area networks (LANs) 202 or other communication networks that supports a plurality of test stations, for example test stations (TS) 216a-c, which are served by one or more LAN servers or computers 205. The test stations 216a-c include test and measurement equipment and may or may not include test station modules (explained below) that receive and send information (e.g., data) to the test and measurement equipment as well as enable communication with the LAN server 205. When test station modules are not included, test and measurement equipment are directly coupled (cabled or wireless communication) to the LAN server 205. The LAN server 205 is coupled to the Internet 210, with or without an intermediary Internet Service Provider (not shown), as is true for other components shown. As is well known to those skilled in the art, the Internet 210 comprises and is coupled to a host of other networks (e.g., LANs, wide area networks, regional area networks, etc.) and users.

The central server and database facility 106 includes a central server 250 that is preferably provided with one or more central databases 230a, and is coupled to the Internet 210, among other networks not shown. Although the database 230a is shown as externally coupled to the central server 250, one skilled in the art would understand that the database 230a can be integrated into the central server 250 in some embodiments. The central server 250 includes performance tracking software (PTS) 252, which supports one or more LAN servers 205 of performance labs 108 that can be provided across many locales. The LAN server 205 can access the central server 250 via browser software, according to well-known mechanisms. Additional information on Internet-based communication and Web-interface generation that may be implemented in the performance tracking system 100a can be found in U.S. patent application Publication No. 2002/0,169,835 A1, published on Nov. 14, 2002, filed on Jan. 18, 2001, and herein incorporated by reference.

In one embodiment, the central database 230a can be maintained and updated, and licensed out for use by one or more users or facilities, such as a corporate or institutional research facility. Access to the central database 230a can be implemented over the Internet 210, or in other embodiments, a local copy can be maintained at the LAN server 205. In the latter embodiment, the LAN server 205 can support the test stations 216a-c, which, for example, may access the LAN server 205 via browser software at each workstation, according to well-known mechanisms.

Further, the mechanisms by which the test stations 216a-c access the LAN server 205 (or the LAN server 205 accesses the central server 250) include CGI (Common Gateway Interface), ASP (Application Service Provider), Java, among others.

One skilled in the art will also understand that the information of the database 230a can be stored on a digital video disc (DVD) or other storage medium. In embodiments where local copies are provided (e.g., local to the LAN server 205), the local databases can be run from the test stations 216a-c, network server 205, etc., and updated periodically or otherwise via the central server 250. Further, one skilled in the art would understand that communication among the various components of the performance tracking system 100a and with the member participants 104 and/or member institutions 102 can be provided using one or more of a plurality of transmission mediums (e.g., Ethernet, T1, hybrid fiber/coax, etc.) and protocols (e.g., via HTTP and/or FTP, etc.).

FIG. 2B is a block diagram of an embodiment of an example central server 250 that can implement the performance tracking software (PTS) 252. With continued reference to FIG. 2A, one skilled in the art will understand that functionality of the example central server 250 can be embodied in the test stations 216a-c and/or LAN server 205 (FIG. 2A), alone or in combination (i.e., in a single component, or distributed over several components), among other embodiments. Further, one skilled in the art will understand that additional components or different components with similar functionality can be included in the central server 250, and/or some components can be omitted, in other embodiments. The performance tracking software 252 can be implemented as an executable program, and may be executed by a special or general-purpose digital computer, such as a personal computer (PC; IBM-compatible, Apple-compatible, or otherwise), workstation, minicomputer, or mainframe computer.

The performance tracking software 252 includes a user-interface (UI) module 254, a statistics processing module 255, and a search engine 257, among other functionality to provide the various performance tracking system features. The user-interface module 254 provides display functions according to well-known underlying display generation and formatting mechanisms. The statistics processing module 255 provides for statistical processing of data, including median, mean, histogram, and/or descriptive statistics, among others, using well-known statistical processing mechanisms. Further, the statistics processing module 255 facilitates data processing integrity. For example, the statistics processing module 255 may detect (and thus alert administrators or others) mean or median shifts of a defined percentage, for example .+—0.5%, on individual or group test data in light of existing cumulative data (e.g., nation-wide data, etc.), which may signal to administrators that the data is of suspect integrity. For example, such variations may signal to administrators that test equipment calibration (e.g., test stations 216a-216c, FIG. 2A) is inaccurate, the test equipment configuration is not set-up properly, and/or that there are equipment problems. From this information, administrators can query test officials, discard the data, and/or adjust the data according to defined specifications, among other actions. The search engine 257 provides database search methodologies according to mechanisms well-known in the art.

If implemented in hardware, as in an alternative embodiment, one or more of the functionality of the performance tracking software 252 can be implemented with any or a combination of the following technologies, which are all well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

Generally, in terms of hardware architecture, as shown in FIG. 2B, the central server 250 includes a processor 260, memory 258, and one or more input and/or output (I/O) devices 270 (or peripherals) that are communicatively coupled via a local interface 280. The local interface 280 can be, for example but not limited to, one or more buses or other wired or wireless connections, as is known in the art. The local interface 280 may have additional elements, which are omitted for simplicity, such as controllers, buffers (caches), drivers, repeaters, and receivers, to enable communications. Further, the local interface 280 may include address, control, and/or data connections to enable appropriate communications among the aforementioned components.

The processor 260 is a hardware device capable of executing software, particularly that stored in memory 258. The processor 260 can be any custom made or commercially available processor, a central processing unit (CPU), an auxiliary processor among several processors associated with the central server 250, a semiconductor based microprocessor (in the form of a microchip or chip set), a macroprocessor, or generally any device for executing software instructions.

Memory 258 can include any one or combination of volatile memory elements (e.g., random access memory (RAM, such as DRAM, SRAM, SDRAM, etc.)) and nonvolatile memory elements (e.g., ROM, hard drive, tape, CDROM, etc.). Moreover, memory 258 may incorporate electronic, magnetic, optical, and/or other types of storage media. Note that memory 258 can have a distributed architecture, where various components are situated remote from one another, but can be accessed by the processor 260.

The software in memory 258 may include one or more separate programs, each of which comprises an ordered listing of executable instructions for implementing logical functions. In the example of FIG. 2B, the software in the memory 258 includes the performance tracking software 252 and a suitable operating system (O/S) 256, such as WINDOWS, UNIX, LINUX, among other operating systems. The operating system 256 essentially controls the execution of other computer programs, such as the performance tracking software 252, and provides scheduling, input-output control, file and data management, memory management, and communication control and related services.

The performance tracking software 252 can be a source program, executable program (object code), script, and/or any other entity comprising a set of instructions to be performed. When a source program, then the program needs to be translated via a compiler, assembler, interpreter, or the like, which may or may not be included within memory 258, so as to operate properly in connection with the operating system 256.

Furthermore, the performance tracking software 252 can be written as (a) an object oriented programming language, which has classes of data and methods, or (b) a procedure programming language, which has routines, subroutines, and/or functions, for example but not limited to, C, C++, Pascal, Basic, Fortran, Cobol, Perl, Java, ASP, and Ada.

The I/O devices 270 may include input devices, for example but not limited to, a keyboard, mouse, scanner, microphone, etc. Furthermore, the I/O devices 270 may also include output devices, for example but not limited to, a printer, display, etc. Finally, the I/O devices 270 may further include devices that communicate both inputs and outputs, for instance but not limited to, a modulator/demodulator (modem; for accessing another device, system, or network), a radio frequency (RF) or other transceiver, a telephonic interface, a bridge, a router, etc.

The performance tracking software 252 also communicates with the database 230a via the local interface 280. As described above, the central database 230a can be external to or integral to the central server 250.

When the central server 250 is in operation, the processor 260 is configured to execute software stored within memory 258, to communicate data to and from memory 258, and to generally control operations of the central server 250 pursuant to the software.

The performance tracking software 252 and the operating system 256, in whole or in part, but typically the latter, are read by the processor 260, perhaps buffered within the processor 260, and then executed.

The performance tracking software 252 can be stored on any computer readable medium for use by or in connection with any computer related system or method. In the context of this document, a computer readable medium is an electronic, magnetic, optical, or other physical device or means that can contain or store a computer program for use by or in connection with a computer related system or method. The performance tracking system 252 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory.

FIGS. 2C-2I are flow diagrams that depict various performance tracking method embodiments provided by the performance tracking software 252 (FIG. 2B). The method steps (depicted in parentheses below) shown and described may occur in other orders, method steps may be omitted, and/or additional method steps may be added while being within the scope of the preferred embodiments of the disclosure. The various method embodiments illustrated in FIGS. 2C-2I will be described in cooperation with some exemplary user interface functional block diagrams, as illustrated in FIGS. 3A-3C. FIG. 3A is a web-site block diagram 300a that illustrates one embodiment of exemplary functionality provided by a web-site provided by the performance tracking software 252 (FIG. 2A) to enable an individual to interact with the performance tracking system 100 (FIG. 1). The blocks in FIG. 3A represent web-interface functionality. Although the blocks are shown interconnected, there is no particular process flow or control hierarchy implied by the interconnections, except as otherwise noted. The web-site block diagram 300a includes, in one embodiment, a user interface 302 which enables user interaction to functional modules that enable access to several categories of information, including benefits to joining 304, registration and edit functionality 306, and newsletter functionality 308. Under the benefits to joining 304, the user can access information about the benefits to being a member 310, the benefits to parents 312, institutions 314, and schools 316. Under the registration and edit functionality 306, the user can register as a member 318, an institution professional 320, a certified tester 322 (e.g., for the performance labs 108, FIG. 1), as well as edit his or her current member profile 324. Under the newsletter functionality 308, the user populates entries with contact information, including entries for email address 326, password, 328, first name 330, middle initial 332, and last name 334.

FIG. 2C is a flow diagram that depicts a method embodiment 252a to enable an individual to register to become a member participant 104 (FIG. 1). With continued reference to FIG. 3A, an individual who wants to become a member can enter the web-site provided by the performance tracking software 252 (FIG. 2A) via the Internet 210 (FIG. 2A) and register (e.g., such as by selecting an icon or entering text (not shown) corresponding to the register as a member functionality 318), including completing a parental consent form (201). Demographics, sports, and other information can be collected via questionnaires. The individual can then print out the parental consent form and payment form (e.g., if the individual is under-aged, the parental consent form may be required) (203). The parent of the individual preferably signs the form and selects a method of payment. The payment form and parent consent documents can be scanned and emailed to the central server and database facility 106 (FIG. 1) (or another location designated by the administrators of the performance tracking system 100, FIG. 1) or mailed to the same by the US Postal service or express carriers such as FedEx, UPS, or other carriers.

After receipt by the central server and database facility 106 (or otherwise by an administrator of the performance tracking system 100, FIG. 1) of the parental consent and payment, the database 230a (FIG. 2A) can be updated to record (and thus activate membership) that the individual is now a member (205). Note that in some embodiments, payment and receipt of consent forms can be automated. An email can be sent to the individual (now a member participant 104, FIG. 1) notifying them that he or she is a member (207). The member participant 104 will preferably be given a temporary password (e.g., valid only for a limited time for security reasons or otherwise) for their initial sign-in. Upon first sign-in, the member participant 104 can be prompted to change his or her password. The member participant 104 can be presented with his or her membership certificate and have access to the member web-site, as represented functionally by the member web-interface block diagram 300b shown in FIG. 3B.

The web-interface block diagram 300b includes, in one embodiment, functionality for a member web user interface 336 that provides for calendar of testing 338, survey data maintenance 340, comparison of certified test results 342, and editing (as well as including viewing mailings and referring a friend options) 344. The web-interface block diagram 300b further includes functionality that provides such information as testimonials 346, trends 348, vendor product survey 350, and parental consent forms 352, as well as information on how to prepare for tests (not shown), among other items.

FIG. 2D is a flow diagram that depicts a method embodiment 252b to enable a member participant 104 (FIG. 1) to register for a test. An email can be sent to the member participant 104 informing him or her of an upcoming certified test and registration information (209). Such an email can be sent responsive to the member participant 104 registering for a test, or unsolicited (e.g., automatically) upon the database 230a (FIG. 2A) being updated with the member participant information (e.g., after parental consent received). Such test information may also be accessed from a web-site (e.g., see calendar of testing 338, FIG. 3B). The member participant 104 can then register for the test and email the registration (211). Alternatively, information on upcoming test dates and sites may be introduced with the initial entry form (e.g., step 203, FIG. 2C), in which case the individual can express an interest at that point as to what dates and times he or she would like to participate in testing. Responsive to receiving the parental consent and updating the database 230a, an email notification of the test date and site can be sent to the member participant 104. Information regarding test registration and information about one or more member participants 104 can be sent to a performance lab 108 (FIG. 1) (213). For example, the information can be downloaded to the test site LAN server 205 (FIG. 2A). The information can include a list of registered member participants 104 for the particular day, time, and location.

FIG. 2E is a flow diagram that depicts a method embodiment 252c to receive and process test data and inform the member participant 104 (FIG. 1) of the results.

Assuming testing has been performed, test information for the member participant 104 can be uploaded from the LAN server 205 (FIG. 2A) to the central database 230a (FIG. 2A) (215). Processing can be performed (217), including statistical processing, monitoring of data integrity (e.g., defined shifts in median or mean scores for a test group and/or individual), grouping of data, etc. The results or an indication that results are available can then be sent to the member participant 104 (219), or the member can access the results (e.g., without notice) shortly after taking the test and manipulate the format of the data to provide comparisons to peer groups, etc., such as by invoking the survey data maintenance functionality 340 (FIG. 3B) in a member participant web-site.

Each successive time, the member participant 104 (FIG. 1) can log-in to a web-site provided by the performance tracking software 252 (FIG. 2A) with a user ID and/or password. The member participant 104 can update his or her personal data as needed via the edit profile functionality 344 (FIG. 3B), and update any of his or her survey data as warranted via the survey data maintenance functionality 340 (FIG. 3B). The member participant 104 may sign up for scheduled certified tests either by selecting a specific site (e.g., performance lab 108, FIG. 1) or search for certified test sites (e.g., via calendar of testing functionality 338, FIG. 3B) that are convenient for them.

FIG. 2F is a flow diagram that depicts a method embodiment 252d to register an institution to become a member. An institution (i.e., an individual(s) representing the institution) preferably registers via the Internet 210 (FIG. 2A) to become a member institution 102 (FIG. 1) (221). A web-site may enable this functionality, as shown in FIG. 3A (register as an institutional professional, 320). An institution will have a background check completed for security reasons. Once the background check is complete, the institution (now a member institution 102) will preferably receive an email with their membership ID and temporary password (223). The member institution 102 may be prompted on first sign-in to change their temporary password.

Note that a similar procedure to that described in FIG. 2F for a member institution 102 (FIG. 1) may also be followed for a coach, athletic director, youth league director, or equivalent. For example, a youth league director undergoes a background check, which is completed for security reasons. The performance tracking software 252 (FIG. 2A) sends the youth league director an email with his or her membership ID and a temporary password. The youth league director is prompted on first sign-in to change their temporary password. The youth league director may be able to download a report on his or her student athletes that have parental consent to provide the test result data.

FIG. 3C is a member institution web-interface block diagram 300c that includes functionality for member institution access. The member institution web-interface block diagram 300c includes, in one embodiment, functionality for a web-interface 354, which enables editing of profiles and viewing of mailings 356, searching 358, provision of a contact results list 360 and vendor product surveys 362. The member institution 102 (FIG. 1) can update their personal data as needed, such as via the edit profile functionality 356.

FIG. 2G is a flow diagram that depicts a method embodiment 252e to enable a member institution 102 (FIG. 1) to search for member participants 104 (FIG. 1) who have criteria in which the member institution 102 has an interest. The performance tracking method 252e preferably provides search screens (e.g., via selection of the detailed search on members functionality 358, FIG. 3C) to the member institution 102 (225). The search screens enable a search on criteria such as demographic, test results, and/or interests. Responsive to receiving the criteria (227), a search is processed (229) (e.g., perform the search, manipulate data, etc.) and data is provided to the member institution 102 (231).

FIG. 2H is a flow diagram that depicts a method embodiment 252f to enable a member institution 102 (FIG. 1) to inform a member participant 104 (FIG. 1) of the fact that there is an interest in him or her by the member institution 102. The member institution 102 (FIG. 1) can review the search results from the search described in association with FIG. 2G. The search result may include a list of participant members 104 that match the search, as identified by an anonymous identification number accessed, for example, via the contact results list functionality 360 (FIG. 3C). If the member institution 102 wishes to contact a participant member 104, they will highlight the corresponding identification number. A member institution 102 can select multiple identification numbers corresponding to multiple member participants 104 on a given search. After selecting one or more identification numbers, the member institution 102 can send in the request (e.g., via email) (233). The performance tracking method 252f can automatically generate a standard email to the member participant(s) 104 informing the same of the interest by the member institution 102 (235). The email sent to the member participant 104 may include a uniform resource locator (URL) for the member institution 102, and/or contact information for the member institution 102.

When the member participant 104 (FIG. 1) receives the email, he or she can review the information and make the decision if he or she wishes to contact the institution. If so, the member participant 104 can directly contact the member institution 102 (FIG. 1) via the contact information provided by the member institution 102. Otherwise, the member can opt-out.

FIG. 21 is a flow diagram that depicts a method embodiment 252g to enable a test administrator to register to apply to become a certified test administrator (CTA) (for the performance lab 108, FIG. 1). A test administrator may access the web-site to register (e.g., register as a certified tester functionality 322, FIG. 3A). A certified test administrator accesses a web-site provided by the performance testing method 252g to obtain registration information (237), which is then responsively provided through the web-site (239). The registration information may include information about courses to take to become certified, and even an on-line course may be provided in some embodiments as administered through by the performance testing software 252. The CTA preferably undergoes a background check completed for security reasons. Once the CTA satisfactorily completes the course, and the background check is approved, the CTA is notified (e.g., via an email) with their membership ID and temporary password (241). The CTA may be prompted on first sign-in to change their temporary password. The CTA can update their personal data as needed. The CTA can download marketing information to advertise certified test dates and locations. The CTA can also schedule certified test sessions for posting on a web-site provided by the performance tracking system 252 (FIG. 2A).

FIGS. 4A and 4B are front elevation and side view schematics, respectively, of an embodiment of a test station module (TSM) 400. One or more test station modules 400 can be disposed at one or more test stations (e.g., test stations 116a-c, FIG. 2A) to communicate information to and from a LAN server 205 located at a performance lab 108, as well as to communicate information to and from test and measurement equipment located at each test station. The test station module 400 includes a handheld module 402 removably disposed in a cradle 408. The handheld module 402 includes a display screen 404 that can be a liquid crystal display (LCD), among others. The display screen 404 provides a mechanism to display the status (e.g., on/off, ready, error prompts) of the test station module 400. The display screen 404 also displays the member participants identification number, test process information, a menu, and messages and/or instructions to a test administrator (e.g., certified test administrator, or CTA). The handheld module 402 also includes a keypad 406 that enables configuration of the test station 116a-1 for a desired test purpose, and includes functionality to start a performance test, monitor performance, and accept results. In other words, the handheld module 402 enables a test administrator to administer a performance test. The test station module 400 includes a barcode scanner 410 to enable scanning of a predetermined identification number of a member participant 104 (FIG. 1). The test station module 400 also includes a banner 412 that can be comprised of practically any material, including vinyl. The banner 412 can be customizable to enable identification of a particular test station (e.g., agility test station). Further included are supports 420 on which the handheld module 402, barcode scanner 410, and banner 412 are supported. In addition, the test station module 400 also includes a light curtain 414 for use in cooperation with electronic/optical test and/or measurement equipment and an embodiment of a controller 416. The controller 416 includes an antenna that enables radio frequency (RF) communication with other devices, such as test and measurement equipment used for testing physical performance.

FIGS. 4C and 4D are front elevation and side view schematics that depict the controller 416 of FIGS. 4A and 4B. The controller 416 includes a display 422 (e.g., light-emitting diode, LED, CD, etc.), key pad 424, and a handle 426. The controller 416 also includes I/O ports 428 for communication with the LAN server 205 (FIG. 2A) and/or with the various test and/or measurement equipment. In other embodiments, the controller and the handheld unit may be incorporated in a single device.

FIG. 4E is a flow diagram that depicts a method embodiment 400a of the test station module 400 shown in FIGS. 4A-4B. The overall control of the test station module 400 resides at the controller 416, and thus the program sequence implied by the method steps are executed by logic of the controller 416. With continued reference to FIGS. 4A-4D, from a ready or activated status (401), the test station module 400 receives the member participant's ID number from the barcode scanner 410 (403). The ID is preferably encoded in the form of a barcode and worn by the member participant 104 (FIG. 1), such as around the member participant's wrist.

Such information can be provided over a cable or wire, or transmitted over air, such as via RF communication. The display 404 of the handheld module 402 presents the appropriate ID number for the member participant 104 (FIG. 1), as well as presents a message alerting the administrator that the test station module 400 is ready to proceed (405). In one embodiment, the ID number may be displayed, while in other embodiments, there may be no display of the ID number. Thus, after the member participant's ID is scanned and authenticated, the CTA may command the member participant 104 to assume the proper starting position for the particular test. Different tests have different starting position requirements for the member participant 104. For example, in an agility test, the member participant 104 may be required to place one of his/her hands at the starting line such that a “break” of an optical beam path is detected (e.g., by a sensor). As another example, flexibility and upper body strength tests may require the participant to sit on or against a template (e.g., such that the legs are positioned at a 30-degree angle and the shoulders/lower back are fully pressed against a wall). Once the CTA is satisfied that that proper starting requirements are met, he/she will press a key on the handheld module keypad 424. Thus, the test station module 400 waits for signals corresponding to selection of one or more of the keys of the keypad 406 (407), and continually waits until an input was received (409). If input was received, the test station module 400 responsively effects an audible sound (e.g., a “beep”) (411). The “beep” signifies to the member participant 104 that he or she is to start the test (e.g., to start running, bending, throwing, etc.). In some embodiments, other forms of alerting the test administrator can be used, such as tactile or visual alarms.

The controller 416 receives and analyzes measurement data and effects the display of the same in the display 404 of the handheld module 402 (413). The handheld module 402 prompts a message on the display 404 to determine whether the results are acceptable (415). If not, operation proceeds to step 405. Otherwise, the handheld module 402 prompts another query to determine whether there is a need or desire for a second opportunity to take the test (417). Test administrators preferably have the ability to reset the test for cause (e.g., someone trips, etc). If so, operation proceeds to step 405, otherwise the controller 416 stores the results in memory and transmits the results to the LAN server 205 (FIG. 2A) preferably when the controller 416 is idle (419), although other times of transmission may be implemented.

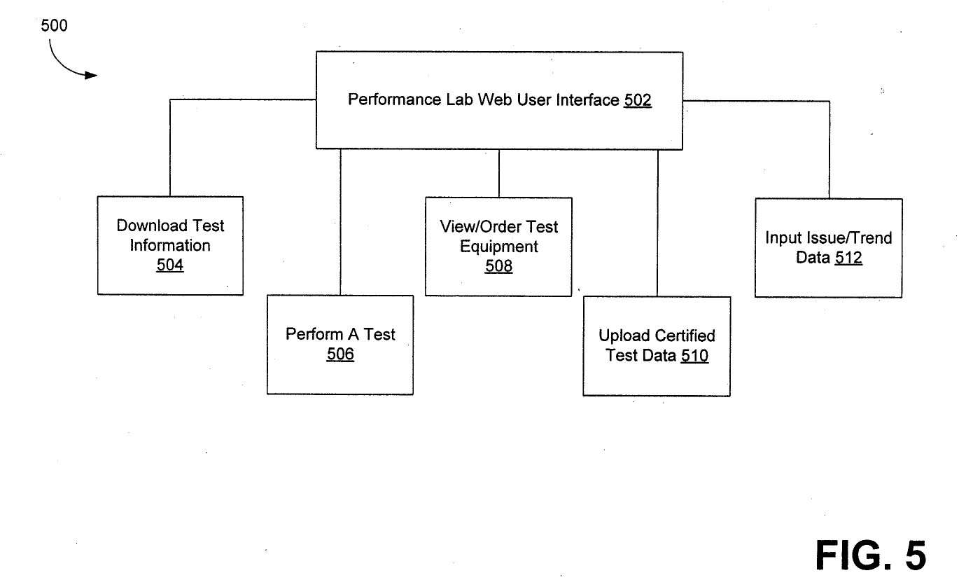

The LAN server 205 (FIG. 2A) communicates the data to the performance tracking software 252 (FIG. 2A) of the central server 250 (FIG. 2A). In one embodiment, the test administrator (or representative thereof), through the use of browser software at the LAN server 205, prompts a web-site provided by the performance tracking software 252 to enable the upload of the certified test data. FIG. 5 is a web-interface block diagram 500 that illustrates functionality for a web-interface embodiment provided by the performance testing software 252. In some embodiments, a user interface with like functionality may be provided by the LAN server 205. As shown, the web-interface block diagram 500 includes functionality for a tester web user interface 502, which enables downloading of test information 504, performing a test 506, viewing and/or ordering equipment 508, uploading certified test data 510, and providing issue/trend data 512.

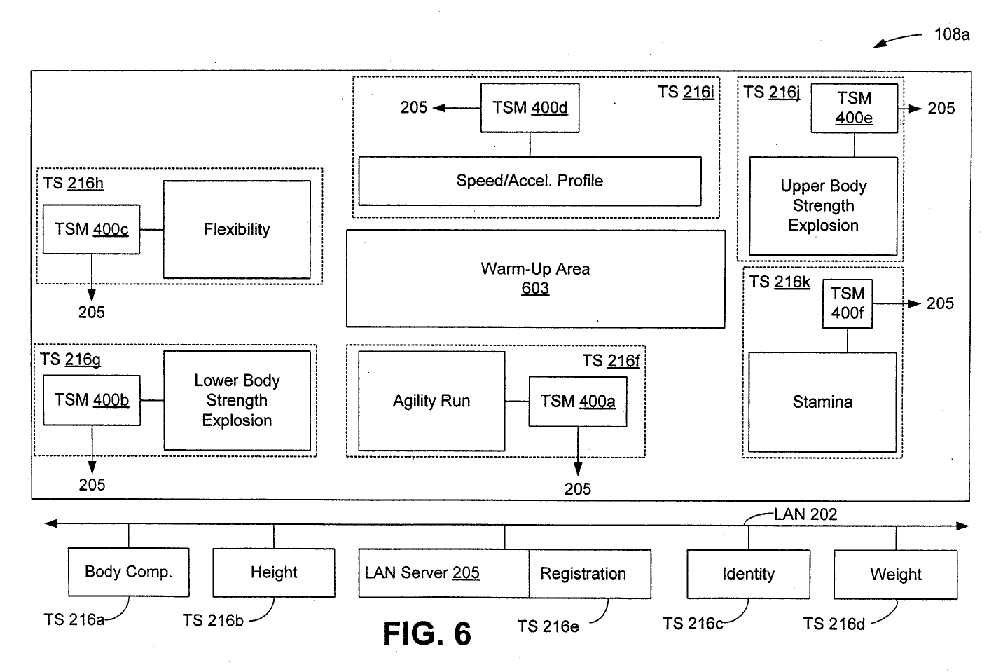

FIG. 6 is a schematic diagram that depicts one embodiment of a performance lab 108a. The performance lab 108a includes test stations 216a (body composition), 216b (height), 216c (identity), 216d (weight), 216e (registration), 216f (agility run), 216g (lower body strength explosion), 216h (flexibility), 216i (speed/acceleration profile), 216j (upper body strength explosion), and 216k (stamina). The test stations 216f-216k may surround a warm-up area 603. Test stations 216f-216k also include test station modules 400a-400f, respectively. The test station modules 400a-400f are coupled to a local area network 202, as are the test stations 216a-216e, which enables communication to and from the LAN server 205. Preferably, the equipment used in the test stations 216a-216k are constructed and operate to a defined standard, which can accelerate testing time and enable the systematic collection of data. In one embodiment, durability, serviceability, and ensured result integrity are important to the design and operation of the equipment, as well as designs that allow for intuitive and efficient operation in a tamper and foolproof package. The equipment is also preferably durable, with the ability to be stored in cases when not in use.

Once registered in the database 230a (FIG. 2A) of the central server 250 (FIG.2A) for a certified performance lab 108a, the member participant 104 (FIG. 1) can arrive at the performance lab 108a and register. The member participant 104 preferably receives an identification media, such as a pre-printed wristband or other article that has the member participant's identification number (e.g., the identification number designated via the performance tracking software 252 during registration) encoded in a barcode on the wristband. That identification number is activated once scanned by a barcode scanner, such as barcode scanner 410 (FIG. 4A). In some embodiments, barcodes may be printed out using a barcode printer (not shown) on-site. To insure integrity, each member participant 104 can utilize the identification media to register at one or more of the test stations 216a-216k.

A certified test administrator at each performance lab 108a can download all registered member participants 104 (FIG. 1) prior to the certified testing session, complete the tests on all member participants 104, and upload test data and other information to the central database 230a (FIG. 2A) via the Internet 210 (FIG. 2A) after the completion of the test regiment. For example, when all member participants 104 have completed their test regiment, all data from each test station 216a-216k can be downloaded into the LAN server 205. After all station data is loaded, the LAN server 205 can be connected to the Internet 210 and all data can be uploaded to the database 230a of the central server 250 (FIG. 2A). Alternatively, data may be “leaked” to the central server 250 throughout testing or periodically during testing, among other mechanisms for data transfer.

In one example implementation, a member participant 104 registers and receives his or her identification media. The member participants 104 progresses through the individual test stations 216a-216k, with the test results being recorded at each station. After one or more tests are completed, the individual test results for each test station 216a-216k are communicated to the LAN server 205. Such communication can occur via a variety of mechanisms, including via a LAN, wireless communication, or a combination of both, among other well-known mechanisms. The results from each test station 216a-216k are compiled at the LAN server 205. Once one or more tests have been compiled at the LAN server 205, the certified test administrator can “upload” the data via the Internet 210 (FIG. 2A) to the central server 250 (FIG. 2A).

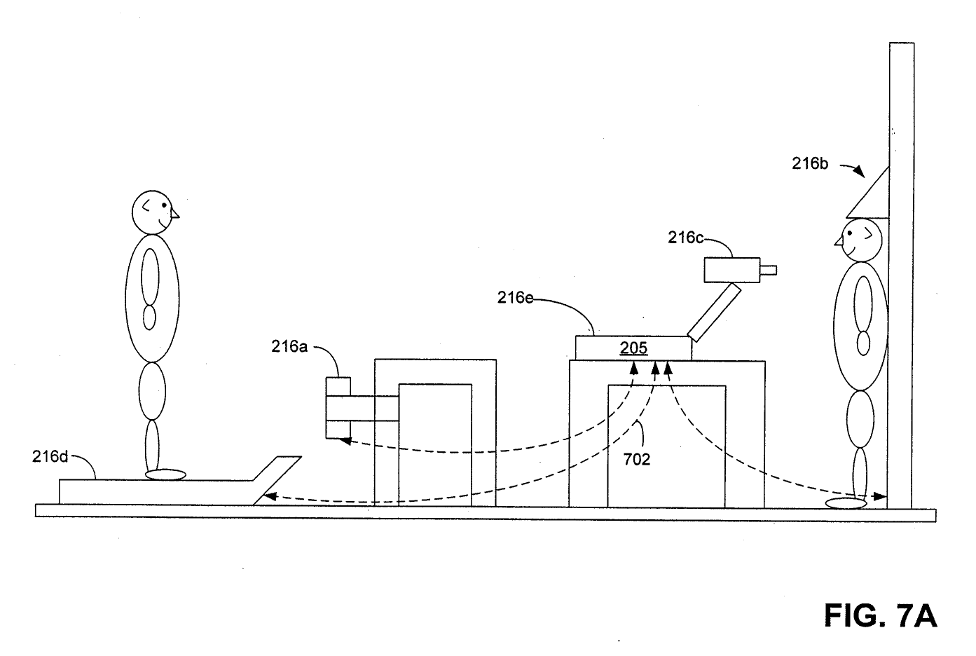

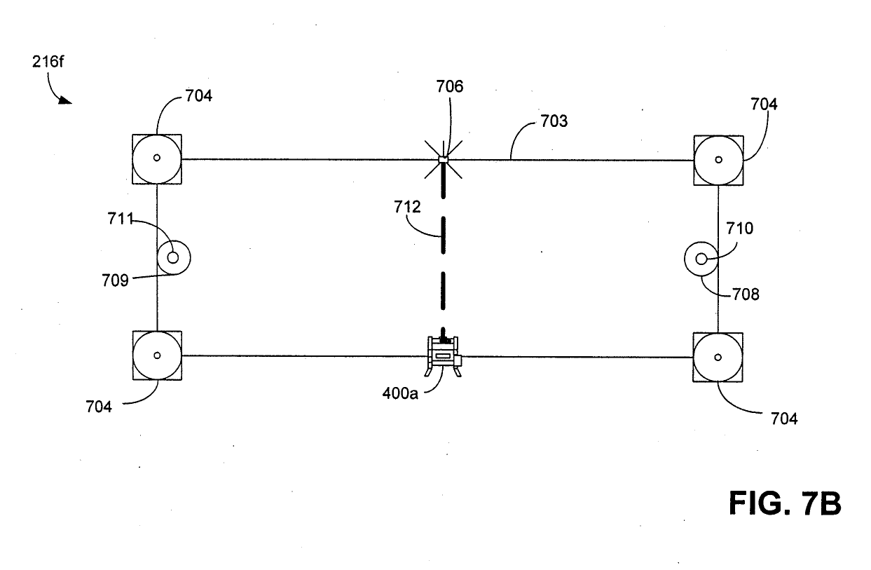

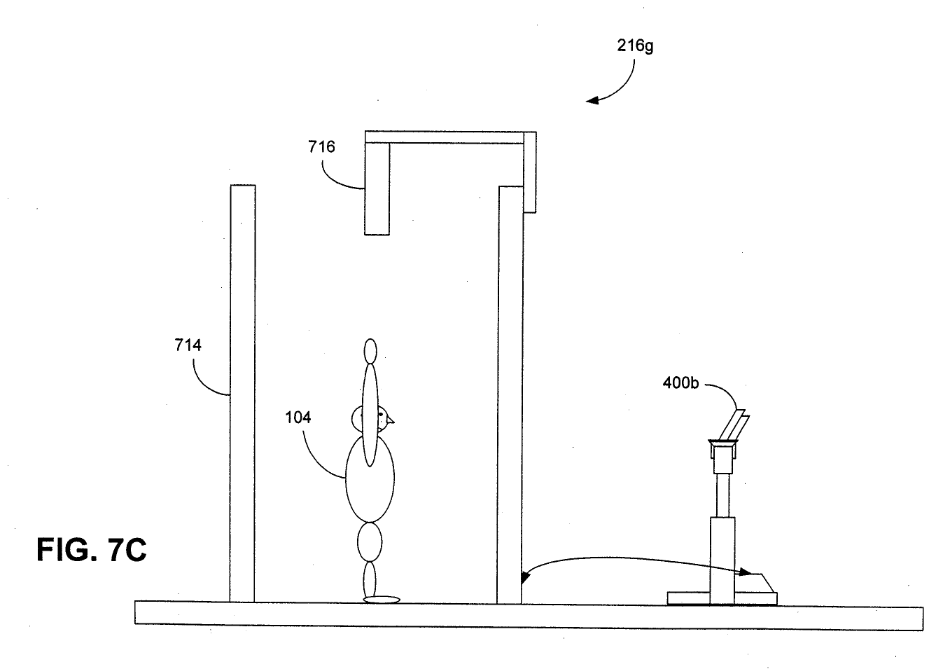

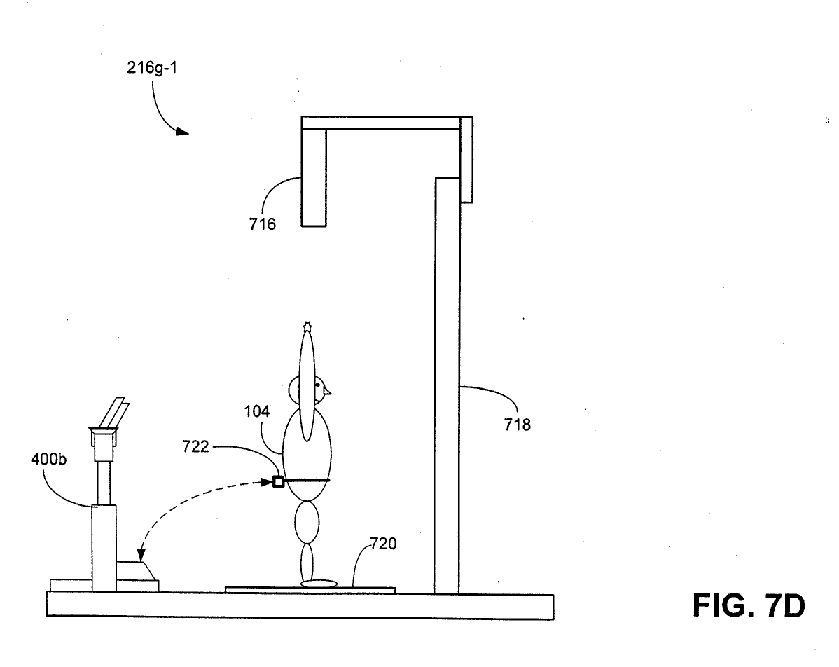

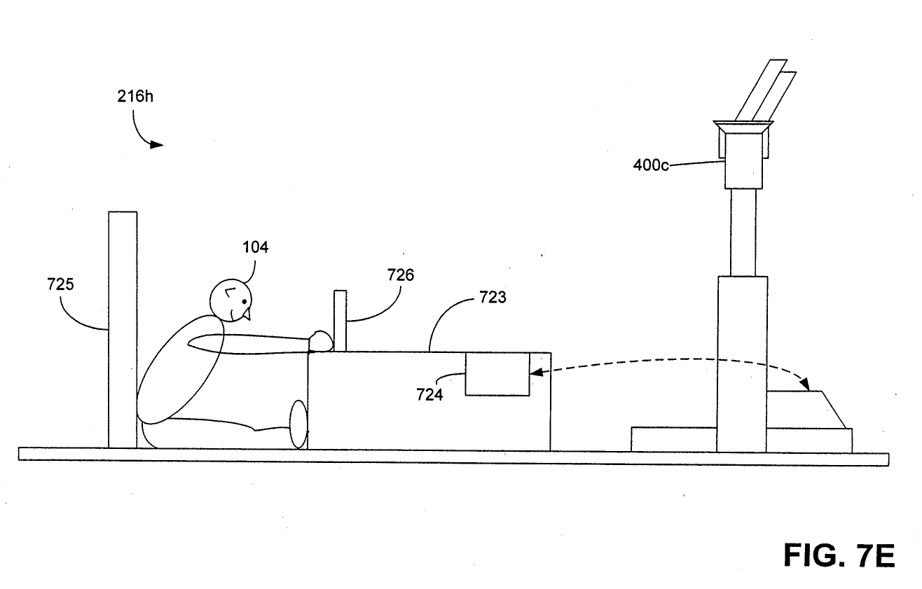

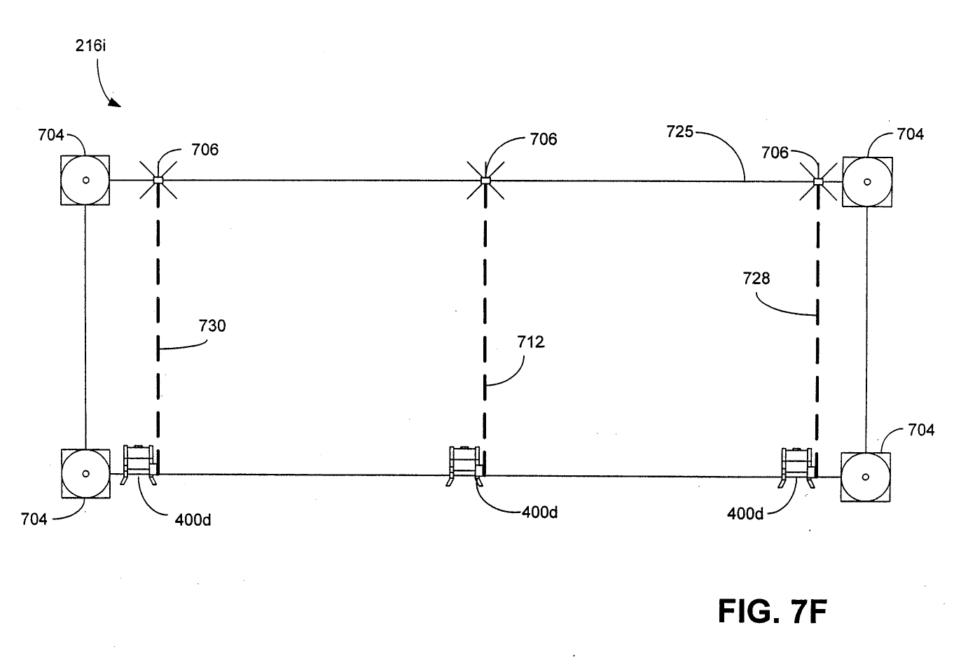

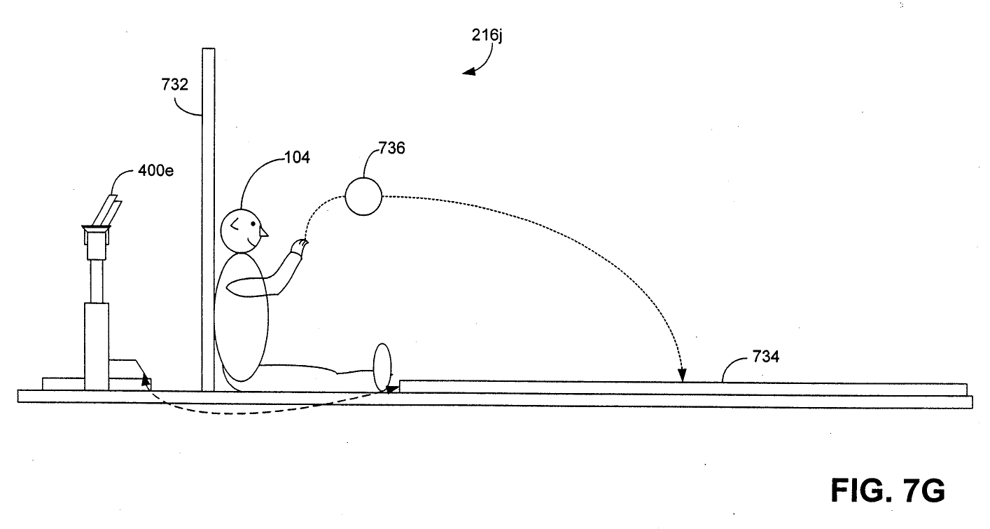

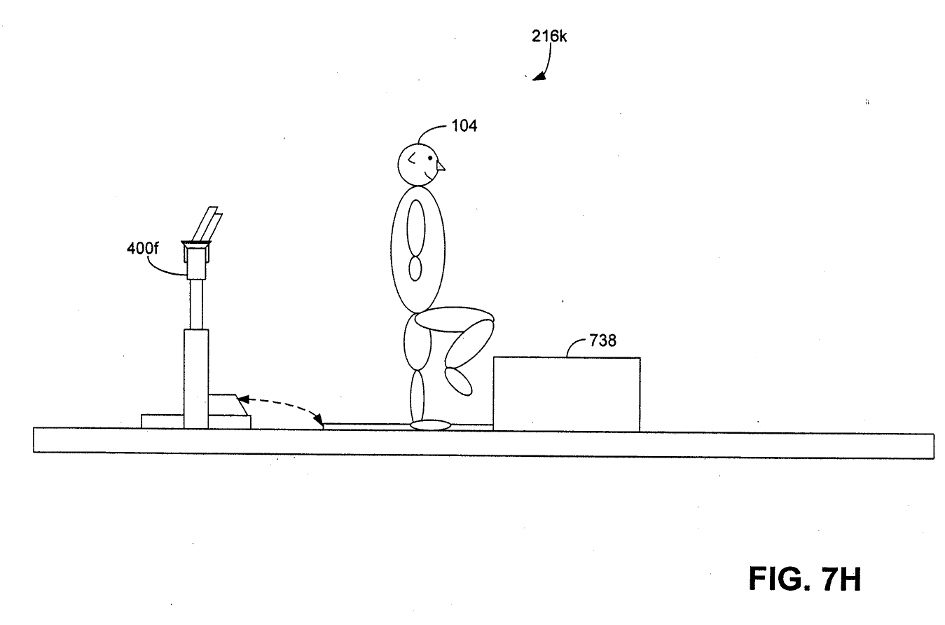

FIGS. 7A-7H are schematic diagrams that illustrate various test station embodiments as generally illustrated in FIG. 6. The performance lab 108a (FIG. 6) provides for physical tests that can be conducted during a student's certified physical performance test. Standardization of the testing regiment (e.g., types of tests, the manner of testing, etc.) is preferred, and as described above, equipment is preferably standardized to enable an acceptable (e.g., acceptable as determined by a recognized standards body or committee) degree of reproducibility and repeatability. A port is preferably provided on each test station module 400 (FIG. 4A) to add an optional digital display. An optional display may be used to show test results to others, such as the member participants 104 (FIG. 1), for instance to provide immediate feedback on results while reducing inquiries to each CTA, or to attract competitive interest. The equipment used in the performance lab 108a, if battery operated, preferably uses extended battery life technology and low battery indicators are preferably provided. All devices preferably have stable technology to eliminate or significantly reduce gymnasium interference (e.g., fluorescent lights, other test station devices, etc.). Alignment indicators are preferred to insure proper set-up (photo-electronic devices, etc.).

It will be understood that the example test stations 216a-216k and tests provided below are not meant to be limiting, and that some tests or test stations may be omitted, additional tests or test stations may be provided, or the described test stations or testing methods may be varied as would be understood in the context of this disclosure by those having ordinary skill in the art. Further, although digital devices are described throughout the disclosure, one skilled in the art would understand that analog technology can also be used, or a combination of digital and analog technology, and be considered within the scope of the preferred embodiments.

FIG. 7A is a schematic diagram that illustrates exemplary test stations 216a-216e. One or more of the test stations 216a-216e may be coupled directly to a LAN server 205, coupled to the LAN server 205 via the LAN 202, or integrated within the LAN server (e.g., registration test station, 216e). The dashed line with double-headed arrows (e.g., 702) represents communication, such as communication between test station 216d and the LAN server 205. Such communication may be enabled via cabling or via wireless technology (e.g., RF, infrared, etc.). Note that cabling is understood to include physical connectivity, such as via coax, hybrid/fiber, among others. Further, one or more of the test stations 216a-216e may be combined in a single device.

Test station 216a is a body composition apparatus, in one embodiment configured as a bioelectric impedance analyzer. The body mass index (BMI) and/or body fat percentage can be measured using the test station 216a or equivalent to determine each member participant's percentage body fat and BMI in relation to his or her age, gender, height, weight, and body build (e.g., youth, athlete, normal). Software in the LAN server 205 preferably automatically populates memory (not shown) in the test station 216a directly from previously recorded height and weight measurements (e.g., measured at test stations 216b and 216d, respectively), in addition to age, gender, and body build acquired from the downloaded registration data (downloaded to the LAN server 205 from the database 230a, FIG. 2A).

Test station 216b is a height measurement apparatus, in one embodiment configured as an electronic height measurement apparatus that includes a slidable disk that can be positioned to rest on a member participant's head and a height scale. A vertical measurement can be taken from the floor to the highest point on the member participant's head. The member participant 104 preferably faces directly ahead with arms by the sides. Shoes should be off, heels together, toes out at an approximately 45-degree angle and turned up with the weight on the heels. The test station 216b may include a foot pad with an outline of the feet pointed at approximately 45 degrees. The member participant's height can be measured to a minimum of approximately the nearest ¼ inch (which can be automatically translated to the metric system within the software of the LAN server 205 or the performance tracking software 252, FIG. 2A, or providing metric units and converts to English units). Other height measurement technology may include a bar code on a wall, laser, infrared, photocell, etc.

Test station 216c is an identity apparatus, in one embodiment configured as a digital camera.

Test station 216d is a weight determining apparatus, in one embodiment configured as a calibrated digital weight scale. Preferably, the member participant's shoes are removed and he or she should be wearing minimal clothing (shorts and T-shirt).

The test station 216d may include a digital readout scale (not shown) that can be used to obtain the member participant's weight to approximately the nearest one pound (which can be automatically translated to the metric system within the software of the LAN server 205 or the performance tracking software 252, FIG. 2A, or provided in metric units and converted to English units).

Test station 216e is a registration apparatus, in one embodiment configured as a software module in the LAN server 205 (although in some embodiments may be configured as a module that is separate from the LAN server 205). The test station 216e can be utilized by the certified test administrator to download those member participants 104 registered to take the certified test. The day of the test, the administrator can simply click and confirm the member participant 104. The member participant 104 can be given an identification media (e.g., using a plurality of methods including but not limited to bar code wrist band or similar technology) that can be used to identify the member participant 104 at each station.

FIG. 7B is a top-plan view of a test station 216f, which includes an agility run set-up in cooperation with a test station module 400a. Note that in some embodiments, the test station modules 400a-400f (for FIGS. 7B-7H) may be replaced, in whole or in part, with acquisition devices such as personal digital assistants (PDAs) or other devices configured with like functionality. The agility run set-up is configured with a running area 703 bordered on four corners with pylons 704 or other marking equipment. At opposite ends of the running area 703 are balls 710 (disposed on mat 708) and 711 (disposed on mat 709). Note that mats 708 and 709 may be rubber mats with a hole in middle for stabilizing the ball. The running area 703 includes a center-line 712, where a reflecting device 706 is located opposite to the test station module 400a. The agility run tests a member participant's ability to change direction laterally and accelerate while maintaining body control and balance. This ability is measured with running tests that require the member participant 104 (FIG. 1) to start, turn, and accelerate.

The agility test can be done using an electronically timed and recorded 20 yard shuttle run. The start from the center position (center-line 712) can be random as the member participant 104 may start to his or her right or left. The member participant 104 preferably places his or her hand on the floor breaking a starting line (center-line 712) that may be marked with an optical beam, or other marking mechanisms. After a specified delay, for example a two (2) second delay, an audible sound (e.g., from the test station module 400a) can let the member participant 104 know that he or she can start when ready. The timer can start when the member participant's hand leaves the starting field. The member participant 104 can break 5 yards, and pick up a ball 710 (e.g., a tennis ball) at mat 710, to register that they have completed a first leg. Then, the member participant 104 breaks back 10 yards crossing the center line 712, picks up a ball 711 at mat 709 to register they have completed the second leg 2. Then, the member participant 104 can run through the center line 712 recording the finish time.

The reflecting device 706 can be disposed, for example, approximately waist high (e.g., via 42-inch tripod mounts), and the test station module 400a can record the finish time. The time recording function inside the controller 416 (FIG. 4A) of the test station module 400a is preferably implemented with fast real-time timing circuitry to enable timing resolution, in one embodiment, to a thousandth of a second. Each participant may have, in one implementation, two opportunities on this test. Therefore, the software of the controller (e.g., controller 416) of the test station module 400a preferably stores two tests or has logic configured to pick the best score. If the controller stores two tests, the best score may be determined by the LAN server 205 (FIG. 2A).

The test station module 400a includes a light curtain 414 (FIG. 4A) for providing light to be reflected from the reflecting device 706, but other technology can be used as well, including a photocell, light emitter/detector module, etc., which can be mounted adjacent to the test station module 400a with or without the support of a mounting apparatus, such as a tripod. Some technology that can be incorporated into, or in cooperation with, the test station module 400a includes laser, infrared, touch pad, etc.

For example, the test station module 400a may use a photocell field or touchpad for starting.

The reflecting device 706 (described for this test and others) may be an optical reflector that reflects light transmitted from the test station module 400a. In some embodiments, recording functionality and/or light beam transmission functionality may be incorporated into a device disposed in place of the reflecting device 706, such that test data is transmitted from the device to the test station module 400a. Some features that may desired on such a device includes the provision for selecting one of multiple frequencies (e.g., 4 position dial and matching on receiving unit to pair transmission frequencies between transmitter and receiver).

The test station module 400a (and/or the reflecting device 706 or equivalent thereof) may also have additional features to improve test conditions, including electronic positioning to insure that equipment will not work without proper location (e.g., 10 yards apart, etc.) (or the provision for a template for proper set-up), minimum of approximately 0.5 mile range, and/or audible sound when field is broken or other alerting mechanisms. Other features may include, for starting position, allowing for the option of either a touchpad or photocell/infrared field, an audible sound incorporated with a 2 second delay when keying up for the start to activate start time (substantially eliminating “touch and go starts”), port to plug in an external stimulus start (light, horn, etc.), minimum RF interference, and capability of indoor or outdoor use.

FIG. 7C is a schematic diagram of a test station 216g, which includes a lower body strength explosion set-up in cooperation with the test station module 400b. The set-up includes two portable stands 714 (or a wall mount with like functionality), with one of the stands configured with an adjustable jump target 716. The test stands 714, in one implementation, may be a minimum of approximately 15 feet tall. The test station 216g is configured as a vertical jump/leg explosion test that can electronically measure and record jump height, using a photo cell field, by computing the difference between the member participant's standing reach and their vertical jump reach to the vertical target 716, thus enabling a vertical jump score. The test stands 714 may be coupled (wireless or cabled) to the test station module 400b. The test stands 714 include sensors or reflectors. Each member participant 104 may have, in one implementation, two opportunities on this test. Therefore, the software of the controller (e.g., controller 416) of the test station 400b preferably stores two tests or has logic configured to pick the best score. If the controller stores two tests, the best score may be determined by the LAN server 205 (FIG. 2A).

Note that in some embodiments, other vertical jump measurement technology may include laser, infrared, photocell field, among others. The member participant's jump distance is preferably measured continuous or a minimum to the ½ inch.

FIG. 7D is a schematic diagram of another test station 216g-1, which includes a lower body strength explosion set-up in cooperation with the test station module 400b. The set-up includes a single portable stand 718 (or wall mount with like functionality) with a jump target 716 attached thereto. The set-up also includes a pressure pad 720, which in some embodiments may be replaced with a photo cell field. An accelerometer 722 is attached to the member participant 104 for determining the vertical jump measurement (or providing data for the determination of vertical jump measurements), with communication between the accelerometer 722 and the test station module 400b occurring via wireless or cabling technology. Each member participant 104 may have, in one implementation, two opportunities on this test. Therefore, the software of the controller (e.g., controller 416) of the test station 400b preferably stores two tests or has logic configured to pick the best score. If the controller stores two tests, the best score may be determined by the LAN server 205 (FIG. 2A).

FIG. 7E is a schematic diagram of a test station 216h, which includes a flexibility test apparatus and a test station module 400c. The flexibility test apparatus is configured as a digital sit and reach box 723, having an adjustment handle 726 and a digital measurement scale 724 that is coupled to the test station module 400d. The digital sit and reach box 723 preferably has a scale (not shown), in inches or centimeters, on the box for immediate feedback to the member participant 104. The flexibility test can measure and score the core body flexibility (i.e., lower back, hips, and hamstring) using the electronic sit and reach box 723 or equivalent with an adjustment handle 726 for arm length. This test can measure each leg independently while the member participant 104 is seated. Although shown with the member participant 104 seated on the floor, in some embodiments, the member participant 104 may be seated on a bench. The flexibility test protocol may require that the member participant 104 must start with his/her lower back and the shoulder tightly against a wall 725. The member participant's flexibility can be measured to approximately the nearest ¼ inch (which can be automatically translated to the metric system within the software of the LAN server 205 (FIG. 2A) or performance testing software 252 (FIG. 2A), or providing in metric units and converting to English units). Other possible technology for measuring flexibility includes the use of bar code, laser, infrared, photocell field, etc.

FIG. 7F is a top plan view schematic diagram of a test station 216i, which includes an acceleration/speed profile set-up and one or more test station modules 400d. The acceleration/speed profile set-up is configured with a running area 725 bordered on four corners with pylons 704 or other marking equipment. Further included in the running area 725 is a center-line 712 and oppositely located end lines 728 and 730. Positioned at the center-line 712 and end lines 728 and 730 are test station modules 400d and measurement devices 706 (mounted, in one implementation, on 42 inch tripod mounts). The acceleration & speed profile testing can be accomplished by the member participant 104 (FIG. 1) running 20 yards (convertible to meters by the LAN server 205 (FIG. 2A) or the performance tracking software 252 (FIG. 2A), or conversion from metric to English). The time is measured electronically (e.g., measured in approximately 1000.sup.th of a second) by the measurement devices 706 from the start (e.g., using a photocell or touchpad, not shown), for instance timed at the 10 yard point and 20 yard point. Therefore, there may be three measurements (2-10 yard split times and total time) for this test. This can enable the profiling of the member participant's ability to start, accelerate and finish a sprint. An option can be offered to the performance lab 108 (FIG. 1) to purchase an additional timing device (e.g., photocell) to measure a 40 yd sprint. This option may necessitate three split times for 10 yards, 20 yards, and 40 yards. The total time and three split times can be recorded. Each member participant 104 may have, in one implementation, two opportunities on this test. Therefore, the software of the controller (e.g., controller 416) of the test station 400b preferably stores two tests or has logic configured to pick the best score. If the controller stores two tests, the best score may be determined by the LAN server 205 (FIG. 2A).

Some embodiments may use other technologies for speed/acceleration measurements, including laser, infrared, photocell, etc. Wireless technology is preferred to eliminate the possibility of tripping hazard. Photocell or touchpad may be used for starting (preferably, a photocell).

FIG. 7G is a schematic diagram of a test station 216j that includes an upper body strength explosion set-up and a test station module 400e. The set-up includes a wall 732 against which the member participant 104 rests and a horizontal electronic field 734 that may be implemented using horizontal laser, infrared, or photocell field technology. The horizontal electronic field 734, in one embodiment, has a minimum distance capability of approximately 20 feet, and may be configured with a floor measurement chart template (not shown). The horizontal electronic field 734 is coupled to the test station module 400e. The upper body explosion and power test can isolate the upper body by placing the member participant 104 in a seated position with their back and hips against a wall. The distance the member participant 104 can throw a medicine ball 736 (e.g., using either a 16 lb. medicine ball or a 12 lb. medicine ball) is measured and recorded electronically. The member participant's legs can be positioned, in one embodiment, at an approximately 30 degree angle through the use of a template in one embodiment, not shown). In a further embodiment, the test station 216j includes a platform/floor upon which a participant can spread their legs. Measurements can be from the wall (e.g., preferably measured continuous or minimum to the approximately ½ inch). Each member participant 104 may have, in one implementation, two opportunities on this test. Therefore, the software of the controller (e.g., controller 416) of the test station 400b preferably stores two tests or has logic configured to pick the best score. If the controller stores two tests, the best score may be determined by the LAN server 205 (FIG. 2A).

FIG. 7H is a schematic diagram of a test station 216k that includes a stamina set-up and a test station module 400f. The set-up includes a floor platform 738 (which may be a standard floor platform) configured with an optical or pressure sensor, and coupled to the test station module 400f. The member participant 104 steps from floor to platform and back. Technology could measure number of steps in a specific time period that can be programmed. The sensors measure (e.g., in approximately 1000th of a second) the frequency the member participant 104 steps from floor to platform. This should be measured continuous. Other possible technology may include one yard or meter horizontal laser, infrared, photocell field, etc.

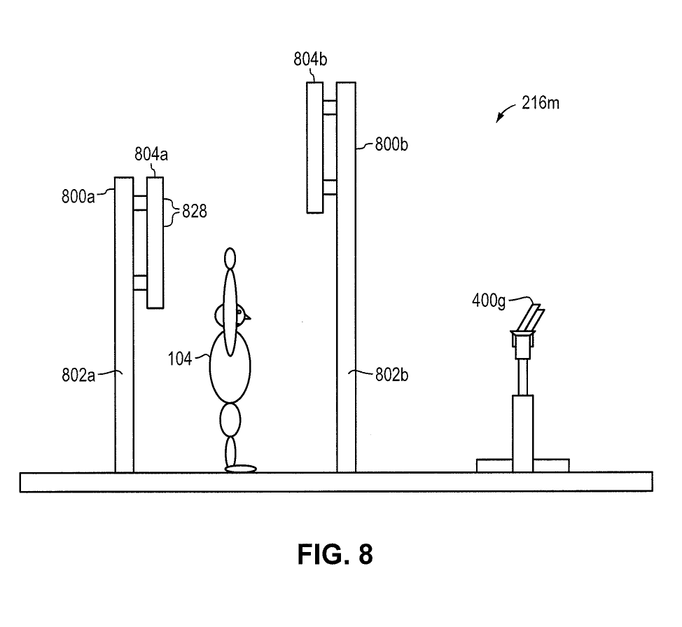

Additional embodiments of test stations which may be used with systems discussed above or with other systems will now be described with reference to FIGS. 8 to 17. FIG. 8 shows a schematic diagram of a test station 216m which may be used to measure lower body strength. The test station 216m may be used in a system in addition to or in place of test stations 216g and 216g-1 discussed above. The test station 216m includes two measuring devices 800a, 800b coupled to a test station module 400g. The test station module 400g may be similar to the test station modules 400 and 400a-400f discussed above with specialized programming to perform functions of the test station 216m. Each of the measuring devices has a height adjustable stand 802a, 802b which supports an electronic device 804a, 804b (e.g., a sensor arrays). In one embodiment, each of the measuring devices 800a, 800b is implemented using the same device. That is, a single measuring device may be employed to provide the functionality of both of the two illustrated measuring devices 800a, 800b, for example, a single longer measuring device may be used. The height adjustable stands are adjustable in height, and as shown in FIG. 8, in the test station, one of the measuring devices 800a is set at a lower height than the other. According to one embodiment, the electronic device includes a number of keys 828 that are similar to the keys of a keyboard. In other embodiments, the electronic devices may be coupled to the same stand and may be oriented with the keys facing in opposite directions. According to one embodiment, a single measuring device is employed in the test station 216m and the measuring device measures the height of the participant's jump and does not measure the participant's reach. In a version of this embodiment, the participant's reach is measured at a different test station.

In various embodiments the electronic devices provide a sensor array, for example, an array of the key-type sensors as illustrated. Further, the electronic devices may be configured to place the sensors in an array having a predetermined geometrical arrangement. For example, in the illustrated embodiment, the keys 828 are arranged in a linear array. Other different geometrical arrangements (such as an arcuate shape or tiered configuration) may be employed. In addition, the structure that supports the electronic devices (e.g., the height adjustable stands) may also be configured to place the sensors in various geometrical arrangements.

In operation, the participant first establishes a baseline height by touching the highest key of the measuring device 800a that the participant can without jumping. Next, the participant jumps and touches the highest key that he/she can on the measuring device 800b. The system records the heights for processing, and can determine the vertical leap of the participant based on the difference between the two measurement points. The heights of the electronic devices are adjusted by adjusting the stands and the particular heights used may be based on characteristics, i.e., age, of the participant pool. The height setting of the stands may be input into the test station module 400g by a test administrator. In other embodiments, the heights may not be adjustable requiring no height input from the administrator.

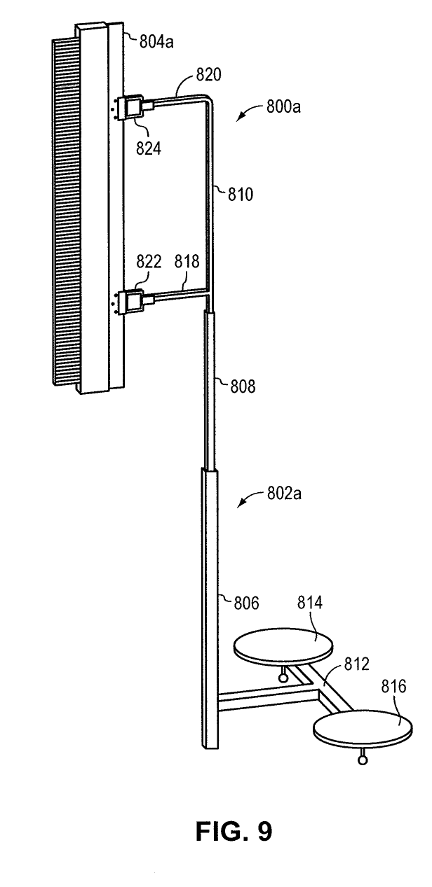

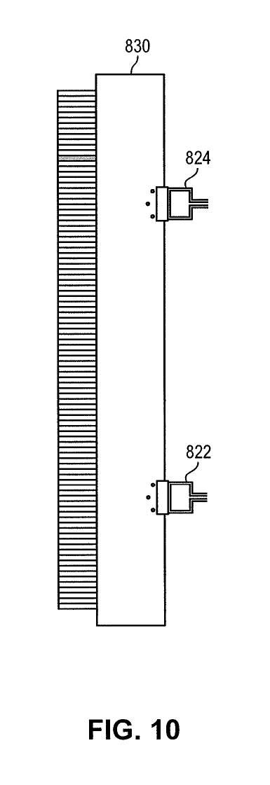

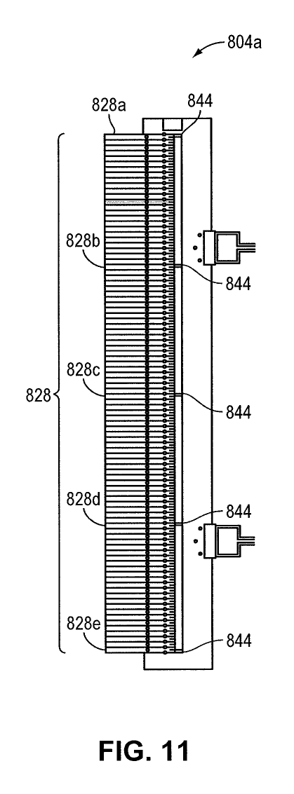

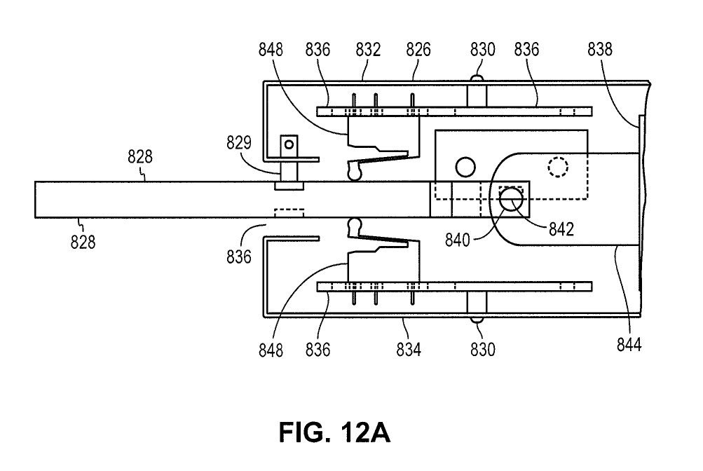

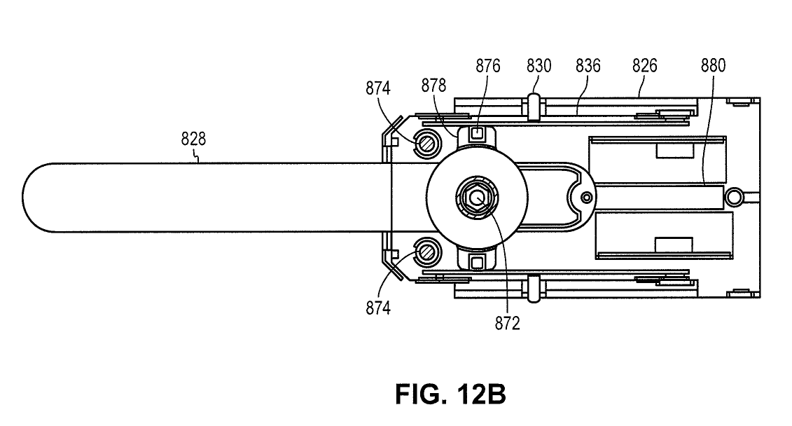

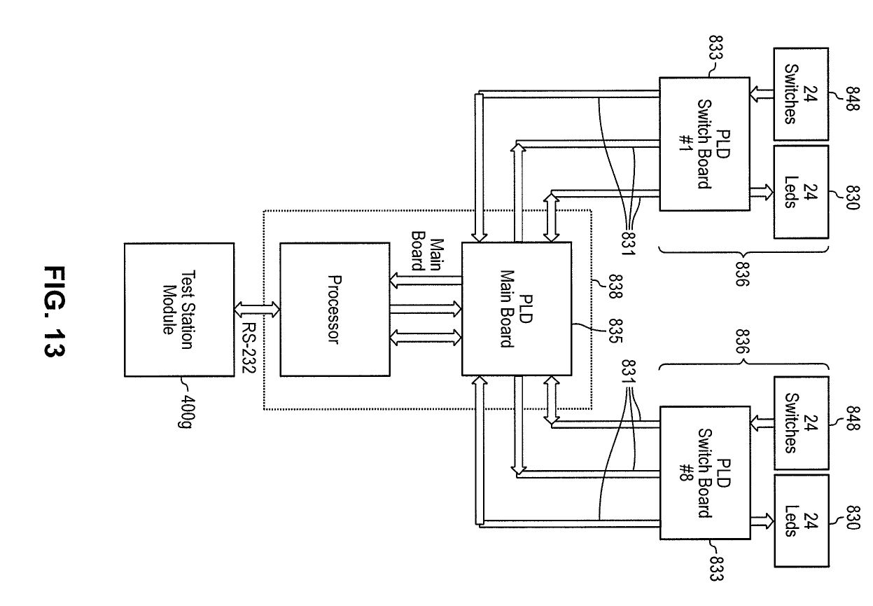

The measuring devices will now be described in more detail with reference to FIGS. 9-12. FIG. 9 shows a more detailed perspective view of one of the two electronic devices 800a. The electronic device 800b is substantially the same as electronic device 800a (e.g., electronic keyboards). Also, while FIGS. 9-11 show only one side of the electronic device, the other side is substantially similar to the side shown. FIG. 10 shows a close up view of one side of the electronic device. FIG. 11 is similar to FIG. 10 with a sheet metal cover panel and four circuit boards removed, and FIGS. 12A and 12B are cross-sectional end views of the electronic device in accordance with two embodiments, respectively. In operation, the keys are moveable in both horizontal directions allowing a person to strike the keys from either side to easily accommodate the use of a participant's left hand or right hand.

The height adjustable stand 802a includes a lower section 806, a middle section 808 and an upper section 810. In one embodiment, the middle section 808 slides into the lower section 806 and the height of the stand can be adjusted be sliding the middle section into or out of the lower section (i.e., a telescoping adjustment). Similarly, the upper section can slide into and out of the middle section to adjust the height of the stand. In the embodiment shown in FIG. 9, the lower section includes a T-shaped support 812 having weights 814 and 816 to prevent the stand from tipping. In another embodiment, a single weight is employed. For example, in one embodiment, the stand 802a includes a weight located to the rear of the stand and includes an additional support located forward of the stand The electronic device is attached at a first end of a bar that extends upward at an angle relative to the floor and is supported by the additional support. The height of the measuring device is adjusted by adjusting the attachment point of the bar to the additional support.

In other embodiments other configurations may be used to support the stand, and in at least one version, the stand may be configured to be mounted directly to a wall or may contain supports that contact a wall for support. The upper section 810 includes two support arms 818 and 820 that support the electronic device 804a using two hinges 822 and 824. The hinges are break-away style hinges that allow the electronic device to rotate about an axis that is parallel to the length of the stand if a participant hits the electronic device with excessive force. The use of break-away hinges helps to reduce the likelihood of damage to the electronic device. In another embodiment, a single break-away hinge is employed in place of the two hinges 822, 824. In one embodiment, the hinges are implemented using hinges available from National Manufacturing Co. of Sterling, Ill. under part number N115-303 V127, although other devices may be used as well. In one embodiment, the stand is made from steel, but in other embodiments, other metals, plastics and composite materials may be used.

The electronic device 804a is contained in a case 826 with keys 828 extending out one side of the case. In one embodiment, ninety-six keys are used along a length of four feet allowing height measurements in half-inch increments, however, in other embodiments, more or less keys providing greater or less increments may be used. The case 826 has a row of holes through which ninety-six light emitting diodes (LEDs) 830 extend. Each diode corresponds to one of the keys, and as described below, during operation, the LED corresponding to the key that was struck by the participant lights and will stay lit until the electronic device is reset. In other embodiments, the LEDs may stay lit until another key is pressed or until the participant completes all of the jumps.

With reference to FIG. 12A, the case 826 has side portions 832 and 834 that are in a clam shell type arrangement forming an opening 836 through which the keys 828 pass. According to one embodiment, the case is made of steel, however, in other embodiments, different metals, plastics, composites or other material suitable for providing a rigid housing are employed. In the embodiment shown, the electronic device includes nine circuit boards including eight device circuit boards 836 and one main controller board 838. Four of the device circuit boards 836 extend along the length of each side of the electronic device.

Each of the keys 828 is coupled to one side portion 832 of the case 826 using a clevis pin 829, and each key has a hole 840 through which a rod 842 (e.g., steel rod) passes to hold the key in place. The rod 842 extends the length of the electronic device and is supported by five brackets 844 that extend from the case 826. Five of the keys (keys 828a, 828b, 828c, 828d and 828e) have slots through which the brackets 844 extend to support the rod 842. In one embodiment, each key is 6⅜ inches long, ½ inch wide and 7/16 inches wide, and is made from polyvinyl chloride (PVC)

Each of the device circuit boards 836 includes 24 switches 848 and 24 LED's. Each of the switches is positioned (as shown in FIG. 12A) such that it is closed when a corresponding key is moved in a direction towards the switch. In one embodiment, the switches are implemented using switches available from Cherry Electrical Products, Pleasant Prairie, Wis. under part no. DA3C-F1RB, and the diodes are implemented using diodes available form Lumex, Inc., Palatine, Ill., under part no. SSL-LX5093SRD/D, however, in other embodiments, other switches and diodes may be used.

The main controller board is contained within the case 826 and is electrically coupled to each of the device circuit boards. The main controller board is coupled to the test station module using a serial interface, for example, an RS-232 or RS-485 compliant interface, however, in other embodiments other schemes, including wireless schemes, may be used to couple the main controller board to the test station module.

Referring to FIG. 12B another embodiment of an electronic device is illustrated. FIG. 12B also illustrates the electronic device in cross-sectional end view. Components that are common to each of the electronic device of FIG. 12A and the electronic device of FIG. 12B are generally not described again with reference to FIG. 12B. According to the illustrated embodiment, the electronic device includes a first rod 872 and a pair of second rods 874 that, in one version extend the full length of the electronic device (e.g., electronic devices 800a and 800b). In one embodiment, the first rod provides a pivot point about which the key 828 rotates. In a further embodiment, the second rods each provide a stop that limits the travel of the key 828 as it rotates clockwise and counterclockwise, respectively, about the first rod.

In accordance with one embodiment, an optical sensor 876 is located in the case 826 to detect a travel of the key 828. In the illustrated embodiment, an element 878 is attached to (or included in) the key 828, and accordingly, rotates about the first rod 872 together with the key 828 when the key is moved by the participant. The rotation of the element 878 is sensed by the optical sensor 876 as the optical path of the sensor is interrupted by rotation of the element 878. That is, in one embodiment, the optical sensor 876 includes a gap through which the element 878 travels when the key 828 is rotated.

As mentioned previously with reference to FIG. 12A, each key 828 includes an associated indicating lamp (e.g., LED 830) that may be connected to a circuit board 836 that is common to a plurality of lamps. Further, FIG. 12B illustrates two optical sensors 876, however, in one embodiment, a single optical sensor 876 is employed with key 828. For example in FIG. 12B, the second illustrated sensor may be employed with an adjacent sensor. In one embodiment, the sensors 830 are arranged such that no two adjacent keys employ sensors located on the same side of the electronic device 800.

In accordance with one embodiment, a spring 880 (e.g., a helical spring) is attached to the key 828 and the case 826. According to this embodiment, the spring 880 provides a force to maintain the key 828 in a neutral position (illustrated) except when it is moved by a participant.

FIG. 13 provides a functional block diagram of the test station 216m showing the major components of the station. In FIG. 13, only two of the eight device circuit boards are shown, however, the other boards are similar to those shown. As indicated in FIG. 13, each of the device circuit boards includes a programmable logic device (PLD) 833 that communicates over data lines 831 with a programmable logic device 835 contained in the main board. In one embodiment, the PLD's are implemented using a device available from Xilinx, San Jose, Calif. under part no. XCR3064XL-10VQ100C, however, in other embodiments, other devices may be used. The main board also includes a processor 837 that in one embodiment is implemented using a device available from Microchip Technology Inc., Chandler, Ill., under part no. PIC18F452-I/PT. The main processor controls the overall operation of the test station 216m and communicates with the test station module 400g.

In one embodiment, the test station 216m is powered from 12 VDC power provided from the test station module. The test station may include various voltage regulators and power supplies to provide other regulated voltages for use in the test station.

The conduct of a performance test using the test station 216m is similar to that for other tests discussed above. Initially, after calibration and setup of the system, a participant scans his/her barcode ID into the test station module. According to one embodiment, the test then begins with the participant touching the highest key that they can reach from a standing position on the measuring device 800a. The LED adjacent to the highest key touched will illuminate. The participant then jumps and touches the highest key that can be reached on the measuring device 800b. Again, the LED associated with the highest key touched will illuminate. In one version, the participant will then be given a second opportunity to touch the highest key possible on the measuring device 800b. Further, in one version, the LED from the first attempt remains illuminated during the second attempt, which can be motivational to the participant to try to exceed the height obtained in the first jump. After the second jump is completed, as with other tests, the operator of the test station will be provided with the opportunity to accept or reject the test results.

The test station 216m above is illustrated and primarily described as employing two different measuring devices to measure the reach of the participant in a standing position, and when jumping, respectively. As readily understood by those skilled in the art, based on this disclosure, in other embodiments, the two devices may be incorporated into one device having an overall measurement range that accommodates both the standing and jumping portion of the test. Alternatively, the standing portion of the test can be completed at a different test station and the hardware associated with the standing measurement may be eliminated from the electronic device. Further, while embodiments discussed above use keyboard like keys as actuation devices, in other embodiments, other types of sensors may be used as actuation devices. Still further, while the electronic devices discussed above have been described for use to measure vertical leap, they can also be used to measure other parameters. Also, while keys are used in measuring devices described above, in other embodiments, optical encoders, or other devices may be used to detect the participant's hand to determine height. The keys may be eliminated in versions of these embodiments.

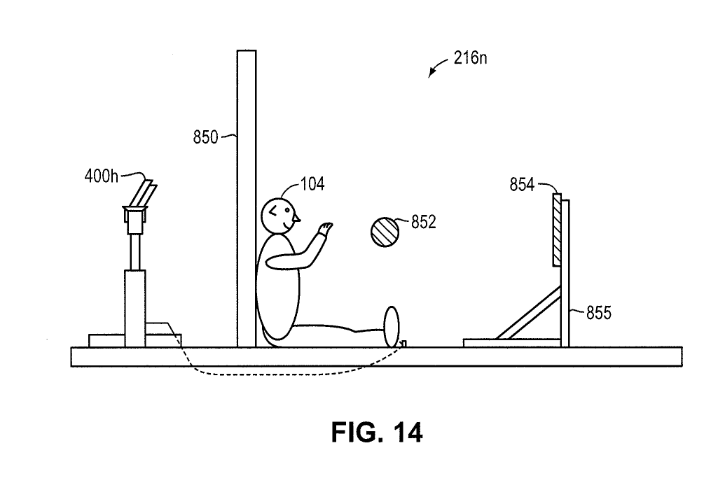

FIG. 14 shows a schematic diagram of a test station 216n which may be used to measure upper body strength. The test station 216n may be used in a system in addition to or in place of test station 216j discussed above. For example, the data collected at the test station 216n may be used to extrapolate a distance that the participant can throw a weighted ball. In one embodiment, the test station 216n includes a wall 850 against which a member participant sits, a medicine ball 852 and a force plate system 854 positioned a horizontal distance from the participant and mounted on a bracket 855 in a manner such that the participant can throw the ball against the force plate. The test station also includes a test station module 400h coupled to the force plate. The test station module 400h may be similar to the test station modules 400 and 400a-400g discussed above with specialized programming to perform functions of the test station 216m. In one embodiment, the force plate is adjustable in the vertical direction to allow the center of the force plate to be aligned with the chest of the participant. In one embodiment, the distance from the wall to the front of the force plate is 61.5 inches, however, other distances may be used as well. In one embodiment, the force plate may be implemented using the same device used to measure weight in the weight test station 216d, however, as discussed below, further processing of the data may be required when measuring upper body strength. In one embodiment, a sixteen pound, nine inch diameter, medicine ball available from D-Ball of Freemont, Calif. under the name Speedball is used, however, in other embodiments, other balls may be used with the system being calibrated for use with such other balls. In addition, various embodiments may employ some other object instead of a ball, and the system may be calibrated for use with the selected object.

In operation, a participant sits against the wall and throws the medicine ball against the force plate. The participant may be given a number of attempts. The force plate records the force of the ball hitting the plate, processes data related to the force, and provides a measurement result to the test station module 400h. As discussed below, in one embodiment, the measurement provided is equal to the kinetic energy imparted to the force plate by the ball.

In another embodiment, the test station 216n includes a seat (e.g., integral to the test station) in which the participant sits when using the test station 216n. In one embodiment, the seat includes a seat back. In a further embodiment the seat is mounted to a frame to which the force plate is also mounted.

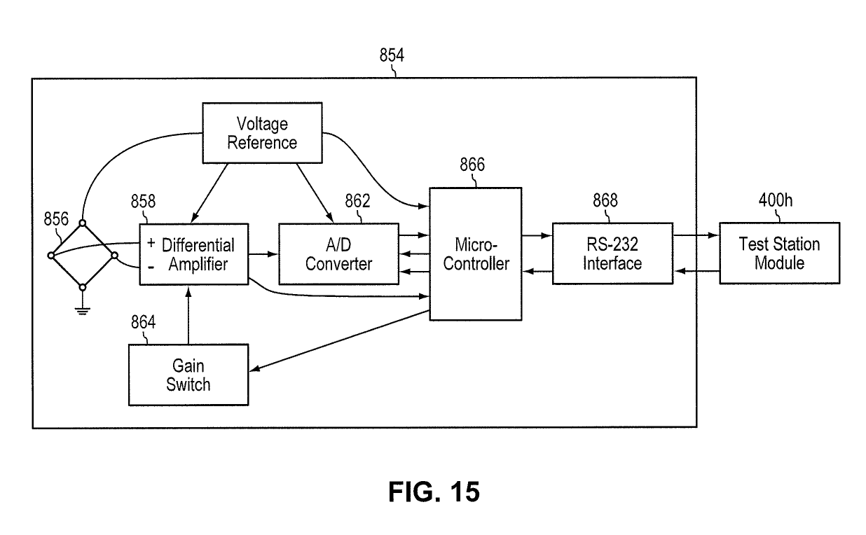

A functional block diagram of the force plate system is shown in FIG. 15. As discussed above, the force plate system may also be used in conjunction with another test station to measure a participant's weight. The force plate system includes a strain gauge bridge network 856, a differential amplifier 858, an A/D converter 862, a gain switch 864, a voltage reference circuit 860, a microcontroller 866 and an interface circuit 868 (e.g., a serial interface circuit).

When the force plate is in use, the bridge network provides an output signal related to the force of impact to the differential amplifier. In one embodiment, the differential amplifier has two stages of amplification. In a first stage, the signal is amplified by a factor of ten, and in a second stage the signal is amplified by either a factor of 10 or 15 depending on whether the force plate is being used to measure weight or upper body force. The setting of the gain of the second stage is set by the gain switch 864 under the control of the microcontroller 866. The output of the amplifier is sampled by the A/D converter. In one embodiment, the sampling rate is 5 KHz and the A/D converter provides a stream of 16 bit digital values to the microcontroller. The interface circuit 868 includes circuitry to provide an interface between the microcontroller and the test station module 400h. The voltage reference circuitry 860 receives five volts from the test station module 400h and provides regulated DC voltages for circuitry in the force plate system.

In one embodiment, the microcontroller is implemented using a Microchip PIC18F252 device having 1.5 KB of RAM, and the RS-232 circuit is implemented using a Maxim 3221 device. In one embodiment, the bridge network is implemented using a device available from Vernier of Beaver, Oreg. under part no. FP-BTA that has been modified to operate with a maximum force of 7000N. In one embodiment, the differential amplifier includes an instrumentation amplifier from Texas Instruments, part no. INA331 and an analog amplifier from Analog Devices, part no. AD9608. Further, the A/D converter is implemented using an Analog Devices AD7684 converter and the voltage reference circuit includes an Analog Devices ADR292 device. In other embodiments, other devices, components and/or circuits may be used to perform the functions described herein.

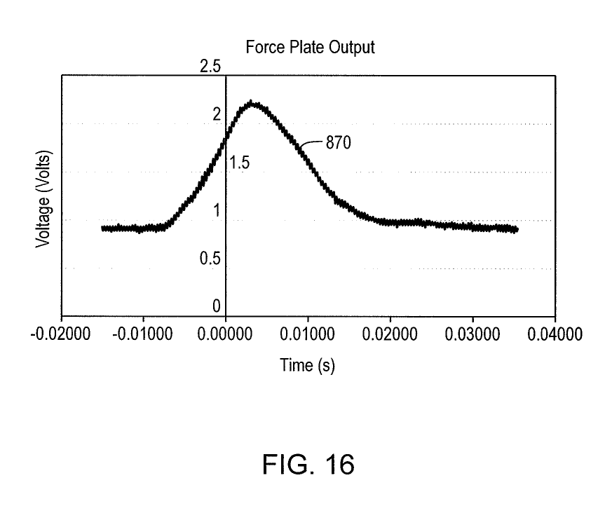

In operation, when the medicine ball is thrown against the force plate, a voltage is provided to the microcontroller. The force applied to the plate from the ball is not instantaneous, but rather will typically be applied over a brief period of time. FIG. 16 provides a curve 870 of voltage vs. time for one example of a sixteen pound medicine ball striking the force plate. To obtain the curve shown in FIG. 16, the ball was supported by a rope and allowed to swing against the force plate in a pendulum type motion. In one embodiment, as will now be described, the kinetic energy imparted to the force plate by the ball is determined for each participant. For each of the voltage measurements (1 measurement at each sample time), an equivalent force is determined. The conversion of voltage to force will depend on the particular bridge network and amplification being used, and modifications may be made to code in the microcontroller to account for other networks. The voltage waveform is converted to a force waveform, which is integrated to obtain the total force. Based on the total force measured and the known mass of the ball, the kinetic energy imparted by the ball may be determined. One or more calibration factors may be applied to account for losses due to vibration of the force plate and inelastic characters of the ball. The calibration factors may be determined by impacting the force plate with the ball being dropped from known heights in a pendulum type manner.

In one embodiment, the kinetic energy for each participant is provided in a signal from the microcontroller in the force plate to the associated test station module 400h. In other embodiments, other parameters, including force and velocity, may be determined and sent to the test station module 400h. Further, calculations to determine force, kinetic energy, velocity or other parameters may be performed in the test station module 400h or main system controller in other embodiments. In at least one embodiment, a medicine ball is thrown by a participant to characterize upper body strength. In other embodiments, objects other than a ball may be used, and in one embodiment, a participant may directly strike the force place or a device coupled to the force plate.

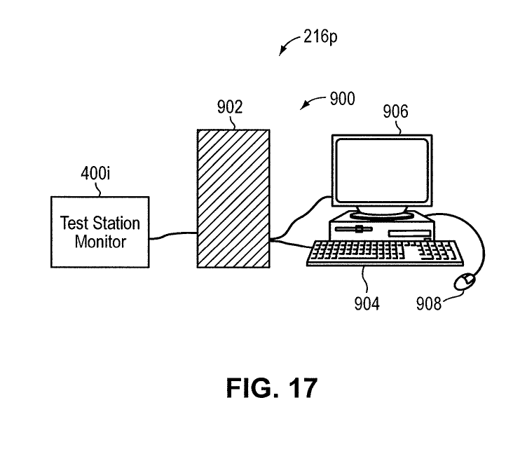

FIG. 17 shows a functional block diagram of a test station 216p that may be used to measure a participant's simple reaction time, recognition reaction time and eye-hand coordination. The test station 216p includes a test station module 400i coupled to a programmed workstation 900. The test station module 400i may be similar to the test station modules 400 and 400a-400h discussed above with specialized programming to perform functions of the test station 216m. The workstation 900 may be implemented using a standard personal computer system such as a desktop or laptop and may include a processor 902, a keyboard 904, a display 906, and a user input device 908, however, in at least one embodiment, a keyboard is not used during the test and may not be included with the workstation during the test. Further, where for example a laptop is employed having an integral keyboard, the keyboard may be “locked out” (e.g., electronically, physically or both) to prevent a participant from entering keystrokes. The workstation may also include headphones or speakers to provide audio outputs. In one embodiment, the user input device is a standard computer mouse, however, in other embodiments, other user input devices may be used, including a trackball with a separate button, a joystick, a trackpad, a touch screen or a data glove. In one embodiment, the user input device 908 includes a console, and in one version, the console includes a track ball and a push button. In one embodiment, programs to perform functions described herein are written in C++, however, in other embodiments, other programming languages and devices may be used.

The test station 216p is used to conduct a number of different tests in which the participant responds to instructions or stimuli on the screen (or through audio outputs) by providing an indication or movement using the user input device 908. In one embodiment, one workstation may be programmed to perform multiple tests, while in other embodiments, each test described below may be performed on different workstations that may be part of different test stations. According to one embodiment, the workstation is employed to perform multiple types of reaction-time and eye-hand coordination tests. In one version of this embodiment, all the tests are performed using visual but not audible stimuli.

In one embodiment, a simple reaction time test is conducted using the test station 216p. In one embodiment, the test station records the time for the participant to react to stimuli on the display 906. The participant may react by pressing a button on the user input device, or in one embodiment, the user starts the test by pressing down a button on the user input device, and during the test, releases the button in reaction to the stimuli. The participant may first log onto the test station using, for example, the user's identification card and a bar code reader associated with the test station. Instructions for the test will then appear on the screen. The user, after reading the instructions, can indicate, using the user input device, that he/she is ready to start the test. In accordance with one embodiment, the test station may be programmed to provide the user with one or more practice rounds to allow the user to become familiar with operation of the test station prior to an actual test run.

In one embodiment, the actual test starts with a “ready” indication on the screen. Once ready, the participant holds down the button on the user input device to start the test. In one embodiment, the system will display “set” after the user presses the button to allow the user to become mentally ready. The screen is then cleared, and a test object is displayed at a random time between 250 and 1500 milliseconds. The participant responds by releasing the button when the object is displayed. The system records the reaction time of the user, that is, the amount of time between a time when the object is first displayed and a time when the participant acknowledges the objects appearance. The user is again instructed to press the button when ready, and the test may then be repeated a number of times. In one embodiment, the test is repeated five times and the test object that is displayed is a circle having a diameter of approximately two inches, however, in other embodiments, other test objects may be used, and the particular choice of test object may be randomized from one test subject to another.

Once the test is completed, the test station calculates a score for the participant. The score may contain an average reaction time, a total reaction time, a best reaction time, and/or other statistical data related to the test. In one embodiment, the score is displayed on the display for the user, however, in other embodiments, the user is not provided with results at the test station. The workstation sends the score to the test station monitor.

In the simple reaction test, the workstation may be programmed to respond to various errors that may occur during the test. For example, if a participant responds before a test object is displayed, then a false reading is indicated, and the test is reset. The test may be configured such that the number of false readings that occur is included in the test results. Also, if the user does not respond within a maximum response time, then an error may be indicated and the test reset.

In accordance with one embodiment, a simple reaction time test is conducted and scored as follows: 1) the system displays “set” after the user depresses and holds the button; 2) after some random time delay between a maximum and minimum amount of time a test object appears on the screen and the “set” graphic disappears (e.g., in one embodiment, the “set” graphic is not cleared in advance of the display of the test object); 3) the participant releases the button when the object is displayed; 4) in one embodiment, the participant's response to the first test object is not used in the scoring (e.g., it may be recorded but not used); 5) the participant continues to respond to at least three subsequent test objects in a similar fashion; 6) each of the participant's responses is recorded and the test is completed following the first three qualifying responses (following the initial response); 7) in one embodiment, a qualifying response is a response that is slower than a minimum response time established for the test; and 8) in one embodiment, the score for the participant is established based on the score of the response having a response time that falls in the middle among the response times for the three qualifying responses (that is, in one embodiment, the score is not an average).

As mentioned in the preceding, in some embodiments, a minimum response time is established. This approach may be used to eliminate response times that are the result of the participant “anticipating” an appearance of the test object. In a version of this embodiment, the minimum response time is 140 milliseconds. Accordingly, in this version, a response that is faster than 140 milliseconds is disqualified. In a further embodiment, the test may be “terminated” where a participant generates a predetermined quantity of disqualifying responses. In one embodiment, such a “termination” automatically generates a “help” flag intended to provide an indication to a test administrator who may then assist the participant in better understanding the test procedure.

In one embodiment, a recognition reaction time test is conducted using test station 216p. The recognition reaction time test may be conducted immediately before or after the simple reaction time test discussed above or may be conducted separately. Conduct of the recognition reaction test is similar to the simple reaction test except that rather than responding to the presence of an object on a display, the participant must first verify that the displayed object is a valid object. The participant will be informed of the identity of the valid test object(s) at the start of the test. In one embodiment, a single type of object is identified as a valid test object, however, other embodiments may include more than one type of valid object. During the test, a number of invalid objects may be displayed, and the user is to respond only when the displayed object is a valid object. The valid object may be the circle described above, and the invalid object may be a square or some other geometric shape. In one embodiment, a number of different invalid objects may be included in the test.

The recognition reaction test may start with an instruction screen and a practice test in a manner similar to the simple reaction test described above. The actual recognition test also starts with “ready and “set” indications on the screen as discussed above. Once ready, the participant holds down the button on the user input device to start the test. The “set” indication appears, the screen is then cleared, and a first object is displayed at a random time between 250 and 1500 milliseconds. In one embodiment, the choice of valid test object verses invalid test object is made on a random basis each time an object is displayed. In another embodiment, a sequence of test objects that includes five valid objects and two invalid objects is used with the order of the sequence varied randomly for each test run.

The proper response by the participant is to ignore invalid objects and to release the button when a valid object is displayed. The system records improper reactions (responding to an invalid object) and the reaction time of the user for valid responses. An invalid object is displayed for a period of time, and then will either automatically disappear, advancing to the next trial, or a prompt will appear requesting the user to release and press the button to proceed to the next trial. The test may be repeated a number of times.

Once the test is completed, the test station calculates a score for the participant. The score may contain an average correct reaction time, a total correct reaction time, a best correct reaction time, and/or other statistical data related to the test including an indication of false reactions. In one embodiment, the score is displayed on the display for the participant, however, in other embodiments, the score is not shown to the participant at the test station. The workstation sends the score to the test station monitor. In a similar manner to the simple reaction test described above, the recognition reaction test may include provisions for responding to false entries and other errors that may occur during the test.

In accordance with one embodiment, a recognition reaction time test is conducted and scored as follows: 1) the system displays “set” after the user depresses and holds the button; 2) after some random time delay between a maximum and minimum amount of time a test object appears on the screen and the “set” graphic disappears (e.g., in one embodiment, the “set” graphic is not cleared in advance of the display of the test object); 3) the participant releases the button when the object is displayed; 4) the participant continues to respond to subsequent test objects in a similar fashion; 5) in one embodiment, the participant's response to the first two test objects that appear is not used in the scoring (further, in one embodiment, one of the first two test objects is a valid test object and the other of the first two test objects is an invalid test object); 6) a response to a total of three valid test objects is required to complete the test; and 7) in one embodiment, the score for the participant is established based on the score of the response having a response time that falls in the middle among the response times for the three qualifying responses (that is, in one embodiment, the score is not an average).

The process described immediately above may also include the following approach in accordance with one embodiment. Here, where a participant responds to an invalid test object, e.g., by selecting the object, a “trial period” is triggered. Responses to valid objects are not included as qualifying responses during the trial period. The trial period ends at the first subsequent point following the erroneous response at which the participant is presented with an invalid test object and does not select that object (e.g., does not respond to the invalid test object). Responses to valid objects following that point are again considered qualifying responses until such time, if any, as the participant again incorrectly responds by selecting an invalid test object. Thus, in one embodiment, these trial periods triggered by the participant's erroneous responses, may serve to prevent the participant from rapidly responding to any test object that appears without taking the time necessary to determine whether it is a valid or invalid object. In a further embodiment, the recognition reaction time test includes a minimum response time as first described above for the simple reaction time test.

In one embodiment, an eye-hand coordination test is conducted using test station 216p. The coordination test may be performed before or after the tests discussed above or may be conducted separately. In one embodiment, the eye-hand coordination test measures the ability of the participant to maintain a controllable object within a randomly moving object on the display screen using a user input device such as a trackball, mouse or joystick.

The test may begin with an instruction screen and a practice test similar to the tests discussed above. During conduct of the test, a first circular object is displayed on the screen with a second smaller circular object centered within the first object. The first circular object is moved randomly around the display screen, and the participant moves the second circular object to attempt to maintain the second object within the perimeter of the first object. In one embodiment, the rate of movement of the first circle is between zero and four inches per second, however, in other versions other rates may be used, and the rate may be varied during the conduct of test, either randomly, or in response to a participant's performance.

According to one embodiment, the distance from the centers of the two circles is measured continuously during the test and is used to determine an overall score of the participant. The actual score may be calculated in a number of ways based on the measured data and may include, for example, an average distance between the two circles or a total of all of the distances measured during the test. In one embodiment, the score is displayed on the display for the participant, however, in other embodiments, the participant is not shown the score at the test station. The workstation sends the score to the test station monitor. In the eye-hand coordination test described above, circular objects are used. In other versions, other shaped objects may be used, with the first object and the second object not having the same shape. In embodiments described above, the test station 216p includes a workstation coupled to the test station monitor 400i. In other embodiments, the functionality of the workstation and the test station monitor may be combined in one device and in one particular version, a bar code reader is coupled to the workstation to allow the participant to register with the workstation, and the workstation includes hardware and software to communication with a central device over a wireless or wired network at a test facility.

In accordance with one embodiment, an eye-hand coordination test is conducted and scored as follows: 1) the test displays two objects for which the participant is to maintain a certain spatial relation, for example, maintain a first moving object within a second object moved by the participant (e.g., electronically moved according to the participant's manipulation of a track ball); 2) the test progresses for a predetermined amount of time, e.g., 30 seconds; 3) a predetermined quantity of data points concerning the spatial relation maintained by the user is recorded during the predetermined time, e.g., 600 data points; 4) a regression method is applied to the data points, e.g., a linear regression; 5) a proportion of variability in the data set provided by the data points is determined, e.g., determine the value of R² for the data points; 6) determine a first score based on an average value of the data points; 7) determine a second score based on a comparison between the R² for the data points and a similar value determined for a test population; and 8) determine the total score by summing the first score and the second score.

According to a further embodiment, the above process may be employed with a test in which the speed at which the first moving object travels around the display screen increases during the test period, i.e., the randomly moving object moves most quickly at the end of the test period.

Any process descriptions or blocks in flow charts described herein should be understood as representing modules, segments, or portions of code which include one or more executable instructions for implementing specific logical functions or steps in the process, and alternate implementations are included within the scope of the preferred embodiments of the present disclosure in which functions may be executed out of order from that shown or discussed, including substantially concurrently or in reverse order, depending on the functionality involved, as would be understood by those reasonably skilled in the art.

It should be emphasized that the above-described embodiments of the performance tracking system 100 (FIG. 1), particularly, any “preferred” embodiments, are merely possible examples of implementations, merely set forth for a clear understanding of the principles of the performance tracking system 100. Many variations and modifications may be made to the above-described embodiment(s) without departing substantially from the principles of the disclosure. All such modifications and variations are intended to be included herein within the scope of this disclosure.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A system for determining lower body strength of a subject, the system comprising:
an electronic device having a plurality of sensors distributed in a predetermined array; and
a frame coupled to the electronic device and configured to support the device to allow the subject to activate one sensor of the plurality of sensors.

2. The system of claim 1, further comprising a controller coupled to the electronic device and configured to receive an indication from the electronic device and determine which of the sensors has been activated during a test of a subject.

3. The system of claim 1, wherein a location of the one sensor relative to others of the plurality of sensors in the array is indicative of the lower body strength of the subject.

4. The system of claim 1, wherein the array includes a vertical arrangement of the plurality of sensors.

5. The system of claim 4, wherein the predetermined array is a linear array.

6. The system of claim 1, wherein the plurality of sensors includes at least one of levers and optical sensors.

7. A method of determining lower body strength of a subject, the method comprising acts of:
distributing a plurality of sensors included in an electronic device in a predetermined array;
detecting activation of one of the plurality of sensors by the subject; and
providing a lower body test result for the subject.

8. The method of claim 7, further comprising an act of locating each of the plurality of sensors such that the location of one sensor relative to others of the plurality of sensors included in the array is indicative of the lower body strength of the subject.

9. The method of claim 7, further comprising an act of arranging the plurality of sensors such that they can be activated by a hand of the user.

10. The method of claim 7, further comprising an act of arranging the array vertically.

11. A system for determining upper body strength of a subject, the system comprising:
an object upon which a force is exerted by the subject during a strength test of the subject;
a frame;
a force detector positionable on the frame to receive the object during the test; and
a controller coupled to the force detector and configured to determine a value related to kinetic energy imparted on the force detector by the object during the test.

12. The system of claim 11, further comprising a communication system coupled to the controller to allow the value to be transmitted to a remote data system.

13. The system of claim 11, wherein the controller is calibrated for a known mass of the object.

14. The system of claim 13, wherein the controller is included in the force detector.

15. The system of claim 14, wherein the force detector includes a plate configured to receive the object during the test.

16. The system of claim 11, wherein the object is propelled by the subject during the strength test.

17. The system of claim 11, wherein an elevation of the force detector is adjustable according to a size of the subject.

18. A method of determining an upper body strength of a subject, the method comprising acts of:
adjusting to a testing position a force detector configured to receive an object upon which a force is exerted by the subject during a strength test of the subject, wherein the testing position is established, at least in part, based on a size of the subject;
detecting a force exerted by the subject; and
providing an upper body strength test result for the subject.

19. The method of claim 18, further comprising an act of calibrating the force detector for a known mass of the object.

20. The method of claim 19, further comprising an act of estimating a distance that the subject can propel the object based on the force.

21. The method of claim 18, further comprising an act of determining a value related to kinetic energy imparted on the force detector by the object during the test.

22. The method of claim 21, further comprising an act of communicating a total force measured to a remote test station module.

23. The method of claim 22, further comprising acts of, with the remote test station module, receiving data concerning the size of the subject and generating information identifying the testing position.

24. A system for evaluating at least one of a participant's reaction time or a participant's eye-hand coordination, the system comprising:
a workstation having a processor, a display and a user input device, wherein the processor is programmed to present one or more objects on the screen, measure a participant's response to presentation of the objects and determine a score for the participant; and
a communication device that communicates the score to a central device in a test facility.

25. The system of claim 24, wherein the workstation is programmed to conduct a recognition reaction time test.

26. The system of claim 25, wherein the workstation is programmed to conduct a simple reaction time test.

27. The system of claim 26, wherein the workstation is programmed to conduct an eye-hand coordination test.

28. The system of claim 24, wherein the workstation is programmed to identify a participant's response that is less than a predetermined minimum response time.

29. A method for evaluating a participant's reaction time, the method comprising acts of:
(a) displaying an object;
(b) recording an input by the participant following act (a);
(c) determining an elapsed time between a time of occurrence of act (a) and a time of occurrence of the input;
(d) repeating acts (a)-(c) for a plurality of objects; and
(e) determining a score based on the elapsed time determined at act (d) for each of the plurality of objects.

30. The method of claim 29, further comprising an act of disqualifying any elapsed time that is less than a minimum response time.

31. The method of claim 29, further comprising an act of eliminating from act (e) an elapsed time determined for an object that is the first object displayed to the participant.

32. The method of claim 29, further comprising an act of determining the score based on a single elapsed time selected from a plurality of elapsed times determined for a plurality of objects, respectively.

33. The method of claim 32, further comprising acts of repeating acts (a)-(c) until a qualifying elapsed time is determined for three objects, and determining a score based on an elapsed time for a first object, wherein a qualifying elapsed time of the first object is less than a qualifying elapsed time for a second object and greater than a qualifying elapsed time for the third object.

34. The method of claim 29, further comprising acts of measuring a recognition reaction time of the participant by displaying either a valid object or an invalid object at act (a); and skipping act (c) for invalid objects.

35. The method of claim 34, further comprising acts of disqualifying a participants response to valid objects displayed subsequent to the participant providing an input at act (b) when an invalid object is displayed.

36. A method for evaluating a participant's eye-hand coordination, the method comprising acts of:
(a) displaying, in a display, a first object and a second object;
(b) allowing a location of the second object in the display to be controlled by the participant;
(c) randomly moving a location of the first object in the display;
(d) collecting data, for a plurality of points in time, representative of a distance between the first object and the second object as the participant moves the location of the second object in an attempt to maintain a spatial relationship between the first object and the second object;
(e) performing regression analysis on the data;
(f) performing an analysis of a variability of the data;
(g) comparing the results of act (f) with benchmark data; and
(h) determining a score based on the results of act (e) and act (g).
